(19) United States
(12) Patent Application Publication
(10) Pub. No.: US 2025/0312488 A1
(43) Pub. Date: Oct. 9, 2025

(54) TARGETED INTEGRATION AT ALPHA-GLOBIN LOCUS IN HUMAN HEMATOPOIETIC STEM AND PROGENITOR CELLS

(71) Applicant: The Board of Trustees of the Leland Stanford Junior University, Stanford, CA (US)

(72) Inventors: Matthew H. Porteus, Stanford, CA (US); Michael Kyle Cromer, Stanford, CA (US); Jessica P. Hampton, Stanford, CA (US); Alvaro Amorin, Stanford, CA (US)

(73) Assignee: The Board of Trustees of the Leland Stanford Junior University, Stanford, CA (US)

(21) Appl. No.: 18/865,511

(22) PCT Filed: May 16, 2023
(86) PCT No.: PCT/US2023/022379

§ 371 Date: Nov. 13, 2024

Related U.S. Application Data
(60) Provisional application No. 63/342,320, filed on May 16, 2022.

(51) Int. Cl. A61K 48/00; A61K 38/44; A61K 38/48; A61P 3/00; A61P 7/04; C12N 5/078; C12N 9/22; C12N 15/11; C12N 15/86; C12N 15/90

(52) U.S. Cl. CPC A61K 48/005; A61K 38/44; A61K 38/4846; A61P 3/00; A61P 7/04; C12N 5/0641; C12N 9/226; C12N 15/111; C12N 15/86; C12N 15/907; C12Y 114/16001; C12Y 304/21022; C12N 2310/20; C12N 2506/1369; C12N 2750/14143; C12N 2800/22

(57) ABSTRACT

The present disclosure provides methods and compositions for genetically modifying hematopoietic stem and progenitor cells (HSPCs), in particular by replacing the HBA1 or HBA2 locus in the HSPCs with a transgene encoding a therapeutic protein.

### CROSS-REFERENCE TO RELATED APPLICATIONS

This application claims priority to U.S. Provisional Application No. 63/342,320, filed May 16, 2022, the disclosure of which is herein incorporated by reference in its entirety for all purposes.

### BACKGROUND

Protein-based therapies such as enzyme replacement therapy (ERT) are a first-line treatment for many genetic disorders. While ERT is effective, it is not curative, and thus patients require expensive and cumbersome lifelong treatment to manage their disease. With advances in genome editing technology in recent years (e.g., CRISPR), there is great interest in developing curative gene therapies for genetic disorders such as hemophilia and phenylketonuria, in order to eliminate the need for lifelong treatment and to improve the duration and quality of life for these patients. Such a cure would require that sufficient levels of expression and activity of the therapeutic protein are achieved, and that editing does not disrupt targeted cell or tissue function.

Phenylalanine Hydroxylase (PAH) is an enzyme that catalyzes the hydrolysis of phenylalanine to form tyrosine. Phenylketonuria (PKU) is a common (approximately 1 in 10,000 births) autosomal recessive PAH deficiency that, if left untreated, can result in a great excess of phenylalanine and in irreversible neurologic damage. In classic PKU, for example, plasma levels of phenylalanine can be greater than 1000 μmol/L, whereas in healthy individuals a typical level is on the order of 80-130 μmol/L.

The current treatment approach for PKU is phenylalanine diet restriction, although this is cumbersome and suffers from poor compliance. Another possible approach includes BH4 supplementation (e.g., Sapropterin) although 70% of patients receive no benefit. ERT is not possible for the treatment of PKU, as PAH is very sensitive to intestinal and plasma proteases. If untreated, PKU can lead to irreversible neurological damage and associated symptoms.

Factor IX is a serine protease that plays a role in the coagulation system. The gene for Factor IX is located on the X chromosome, and mutations can lead to an X-linked deficiency called hemophilia B. Hemophilia B can be a severe disease, with around 30% of patients dying from a bleeding episode. Hemophilia B is considered severe if the individual has less than 1% of normal FIX activity, moderate with 1-5% normal FIX activity, and mild with 5-30% activity. Current treatment for hemophilia B is enzyme replacement therapy (ERT), although this approach is both costly and cumbersome, as it needs to be performed 2-3 times/week and can cost from $300K-500K per year.

Certain current gene therapy approaches have several important limitations that must be noted, including the presence of pre-existing neutralizing antibodies (NAbs) to adeno-associated viruses (AAV) in up to 60% of the adult population, meaning a large proportion of patients are ineligible. Indeed, data shows even low levels of NAbs can compromise treatment efficacy. Durability of transgene expression in trial patients is another concern, leading the FDA to recently reject approval of a hemophilia A gene therapy because of phase 1/2 and 3 data showing a continued decline in FVIII activity levels over 0.5-3 years of follow up. Re-dosing of patients for whom efficacy wanes over time is not currently feasible, as systemic administration of AAV elicits the development of NAbs that are maintained at high levels for tip to 15 years following treatment, with cross-reactivity to multiple AAV serotypes.

There is thus a need for new, safe and effective approaches for introducing therapeutic genes such as PAH and FIX into the red blood cells of individuals with PKU or hemophilia B. The present disclosure satisfies this need and provides other advantages as well.

### BRIEF SUMMARY

The present disclosure provides methods and compositions for genetically modifying hematopoietic stem and progenitor cells (HSPCs), in particular by replacing the HBA1 or HBA2 locus in the HSPCs with a transgene encoding a therapeutic protein.

In some aspects, the present disclosure provides a method of genetically modifying a hematopoietic stem and progenitor cell (HSPC) from a subject, the method comprising:
introducing into the HSPC a guide RNA targeting the HBA1 or HBA2 locus, an RNA-guided nuclease, and a homologous donor template comprising a transgene encoding Factor IX, wherein
the RNA-guided nuclease cleaves the HBA1 or HBA2 locus, but not both, in the cell; the transgene is integrated into the genome by homology directed recombination (HDR) at the site of the cleaved HBA1 or HBA2 locus; and the integrated transgene directs the expression of Factor IX in the HSPC; and wherein
   the Factor IX comprises an exogenous signal peptide;
   the Factor IX comprises two or more amino acid substitutions relative to the wild type sequence shown as SEQ ID NO: 13, wherein the two or more amino acid substitutions are selected from the group consisting of R318Y, R338E, R338L, and T343R; and/or
   the transgene comprises a truncated intron 1 of the FIX gene.

In some embodiments, the method further comprises isolating the HSPC from the subject prior to the introducing of the guide RNA, RNA-guided nuclease, and homologous donor template. In certain embodiments, the target sequence of the guide RNA comprises the sg5 target sequence (SEQ ID NO:1), and the RNA-guided nuclease cleaves the HBA1 locus. In particular embodiments, the homologous donor template comprises an HBA1 left homology arm comprising the sequence of SEQ ID NO:3 or a subsequence thereof, and/or HBA1 right homology arm comprising the sequence of SEQ ID NO:4 or a subsequence thereof. In certain other embodiments, the target sequence of the guide RNA comprises the sg2 target sequence (SEQ ID NO:2), and the RNA-guided nuclease cleaves the HBA2 locus.

In some embodiments, the subject has hemophilia B, and the genetically modified HSPC expressing Factor IX is reintroduced into the subject. In some embodiments, the reintroduction of the genetically modified HSPC into the subject improves one or more symptoms of the hemophilia B.

In some embodiments, the expression of the integrated transgene is driven by an endogenous HBA1 or HBA2 promoter. In other embodiments, the expression of the integrated transgene is driven by an exogenous promoter. In some instances, the exogenous promoter is the SFFV promoter.

In some embodiments, the integrated transgene replaces the HBA1 or HBA2 coding sequence in the genome. In some embodiments, the exogenous signal peptide is an IL6 signal peptide. In some embodiments, the transgene further encodes a truncated EPO receptor (tEPOR) downstream in fusion with the Factor IX. In some embodiments, the tEPOR is linked to the Factor IX through a T2A peptide sequence.

In some embodiments, the amino acid substitutions comprise R318Y, R338L, and T343R. In some embodiments, the intron 1 is truncated by at least about 0.5, 1.0, 1.5, 2.0, 2.5, 3.0, 3.5, 4.0, 4.5, 4.6, 4.7, 4.8, 4.9, 5.0, 5.5, 5.6, 5.7, 5.8, 5.9, 6.0, or more kb relative to the full-length FIX intron 1. In some instances, the intron is truncated by about 4.8 kb or about 5.9 kb. In some embodiments, the transgene is codon optimized.

In some embodiments, the transgene comprises a sequence shown as SEQ ID NOS: 6-11 or a subsequence thereof, or a nucleotide sequence comprising at least about 70%, 75%, 80%, 85%, 90%, 95%, 96%, 97%, 98%, 99%, or more identity to any of SEQ ID NOS: 6-11 or a subsequence thereof, wherein the Factor IX encoded by the transgene comprises two or more amino acid substitutions selected from the group consisting of R318Y, R338E, R338L, and T343R. In certain embodiments, the Factor IX encoded by the transgene comprises the amino acid substitutions R318Y, R338L, and T343R.

In some embodiments, the guide RNA comprises one or more 2′-O-methyl-3′-phosphorothioate (MS) modifications. In some instances, 2′-O-methyl-3′-phosphorothioate (MS) modifications are present at the three terminal nucleotides of the 5′ and 3′ ends. In some embodiments, the RNA-guided nuclease is Cas9. In some instances, the Cas9 is a high fidelity Cas9. In some embodiments, the guide RNA and the RNA-guided nuclease are introduced into the HSPC as a ribonucleoprotein (RNP) by electroporation. In some embodiments, the homologous donor template is introduced into the cells using a recombinant adeno-associated virus (rAAV) serotype 6 vector.

In some embodiments, the method further comprises a step in which the genetically modified HSPC is induced to differentiate in vitro into a red blood cell (RBC). In particular embodiments, the subject is a human.

In some aspects, the present disclosure provides a FIX transgene comprising a sequence shown as any of SEQ ID NOS: 6-11 or a subsequence thereof, or a nucleotide sequence comprising at least about 70%, 75%, 80%, 85%, 90%, 95%, 96%, 97%, 98%, 99%, or more identity to any of SEQ ID NOS: 6-11 or a subsequence thereof, wherein the Factor IX encoded by the transgene comprises two or more amino acid substitutions selected from the group consisting of R318Y, R338E, R338L, and T343R relative to SEQ ID NO:13.

In some embodiments, the Factor IX comprises the amino acid substitutions R318Y, R338L, and T343R In some embodiments, the transgene comprises an IL6 signal peptide. In some embodiments, the transgene comprises a truncated intron 1 of the FIX gene.

In some aspects, the present disclosure provides an HSPC comprising the FIX transgene described herein. In some embodiments, the FIX transgene is integrated into the HSPC genome at the HBA1 or HBA2 locus, but not both. In some embodiments, the HSPC was modified using the method described herein.

In some aspects, the present disclosure provides a red blood cell produced by inducing the differentiation in vitro of the genetically modified HSPC described herein into a red blood cell.

In some aspects, the present disclosure provides a method of genetically modifying a hematopoietic stem and progenitor cell (HSPC) from a subject, the method comprising:
introducing into the HSPC a guide RNA targeting the HBA1 or HBA2 locus, an RNA-guided nuclease, and a homologous donor template comprising a PAH transgene, wherein
the RNA-guided nuclease cleaves the HBA1 or HBA2 locus, but not both, in the cell; the transgene is integrated into the genome by homology directed recombination (HDR) at the site of the cleaved HBA1 or HBA2 locus; and the integrated PAH transgene directs the expression of phenylalanine hydroxylase in the HSPC; and wherein
the PAH transgene comprises the sequence shown as SEQ ID NO:5.

In some embodiments, the subject has phenylketonuria, and the genetically modified HSPC expressing phenylalanine hydroxylase is reintroduced into the subject. In some embodiments, the reintroduction of the genetically modified HSPC into the subject improves one or more symptoms of the phenylketonuria. In some embodiments, the method further comprises administering BH4 to the subject.

### BRIEF DESCRIPTION OF THE DRAWINGS

FIG. 1A: Highlighting the base pair (bp) mismatches between the HBA1 and HBA2 loci at the sg5 binding site. FIG. 1B: The sg5 guide targets HBA1 but there is a 5 bp mismatch at the same site in HBA2. FIG. 1C: The indel rates created by 5 different guides targeting various regions in HBA1 or HBA2 were tested and compared. In the right-most column of this graph, the sg5 guide had an indel rate of about 80% in HBA1 with essentially 0 off-target cutting in HBA2. FIG. 1D: Overview of the methods described herein to engineer and optimize RBC-specific therapeutic protein expression (e.g., by systemic therapeutic enzyme secretion or circulation of RBCs acting as mobile therapeutic enzyme reservoirs). Using a combined AAV6/Cas9 ex vivo approach, HSPCs can be targeted for replacement of HBA1 with the transgene of interest, leaving the inserted transgene under control of the endogenous HBA1 promoter. Upon differentiation, progeny in the erythroid lineage expressing alpha-globin will also produce high levels of the therapeutic transgene for either cytoplasmic expression or secretion (depending on the transgene and vector design).

FIGS. 2A-2G: To determine whether RBCs are capable of expressing PAH at high levels in vitro, human CD34⁺ cells were targeted with an AAV6 vector carrying a codon-optimized PAH construct for targeted replacement of HBA1 with the PAH transgene. Cells were differentiated in vitro and analyzed for targeting rates by ddPCR, RBC differentiation by flow cytometry, and PAH expression by western blot. FIG. 2A: Vector design and integration scheme: The delivery PAH vector contains codon-optimized PAH cDNA flanked by Homology Arms (HA) for HBA1: Left HA=466 bp including the 5′UTR of HBA1; right HA=400 bp including 98 bp of the 3′UTR of HBA1 and an additional 302 bp of downstream sequence. The delivery PAH-YFP vector contains a constitutive SFFV promoter immediately upstream of the PAH cDNA, and a T2A (self-cleaving peptide) sequence and YFP cDNA inserted immediately downstream (before the stop codon). Upon translation, the T2A peptide functions as a ribosomal-skipping site which terminates and then re-initiates translation, resulting in the production of 2 distinct proteins from the sequences up- and downstream of the skipping site. Thus, SFFV will drive co-expression of PAH and YFP. FIG. 2B: In vitro targeting and differentiation protocol: CD34⁺ HSPCs are thawed and cultured for 2 days before targeting. As previously described (Bak et al., Nat Protocols, 2018), Hi-fidelity SpCas9 protein is pre-complexed with sgRNA targeting a locus in the 3′ UTR of human HBA1 (Cromer et al., Nat Med, 2021). Cells are electroporated for delivery of the Cas9/sgRNA complex (RNP) into the nucleus, which makes a cut at the target site in the genome. This is immediately followed by addition of AAV6 carrying the PAH delivery construct (at an MOI of 5000) to the culture which facilitates homology-directed repair (HDR) of the RNP-induced break, leading to replacement of the HBA1 with the transgene as described above. 2 days after targeting RBC differentiation is initiated, using a 14-day, 3 phase protocol. FIG. 2C: Targeting Analysis: At day 0 of RBC differentiation (2 days post-targeting) and day 14 (16 days post-targeting), cells are harvested for allelic targeting analysis by ddPCR (MOCK=electroporated only; RNP=electroporated+Cas9/sgRNA complex; AAV=electroporated+AAV6; RNP+PAH=Electroporated+Cas9/sgRNA complex+AAV6). FIG. 2D: RBC Differentiation analysis by FACS: At day 14 of RBC differentiation cells are harvested for FACS analysis of cell surface markers of erythroid differentiation. Here we have reported the percentage of CD34⁻/45⁻ cells that are GPA⁺/CD71⁺, demonstrating that targeting does not negatively impact the ability of HSCs to differentiate down the erythroid lineage. FIG. 2E: PAH Western blot: At day 14 of RBC differentiation, cells were also harvested for qualitative analysis of protein expression by western blot targeted with either HBA1-PAH or SFFV-PAH-T2A-YFP. Compared to the GAPDH reference protein, high levels of PAH expression in both the HBA1-PAH and SFFV-PAH-T2A-YFP vectors are observed in the bulk population of targeted cells post-differentiation. To validate the efficiency of T2A, the relative quantity of PAH-YFP fusion protein (˜78 kD) vs. PAH (51 kD) was evaluated by measuring the intensity of the two bands in the upper portion of the “PAH-T2A-YFP” column in the Western blot image. Roughly 98.9% of detected PAH protein was present in the correct “cleaved” form, suggesting that the T2A peptide sequence is highly efficient and does not meaningfully interfere with PAH expression. FIG. 2F: In vitro PAH enzymatic activity assay. HBA1-PAH showed significantly more PAH activity than SFFV-PAH-YFP (p<0.001 for both Bulk and Sorted), suggesting the HBA1 promoter is a robust driver of protein expression in erythroid cells, even for non-erythroid proteins such as PAH. FIG. 2G: % YFP+ of live cells at d14: HSCs were targeted with SFFV-PAH-YFP and differentiated into RBCs as previously described. On d7, cells were sorted for YFP, and both Bulk (unsorted) and Sorted cells were returned to culture. At d14 of RBC diff, cells were harvested and analyzed by FACS for % YFP+ of live cells (aka, % edited cells). Connecting lines indicate unique replicates (donors). The sample denoted with an asterisk (*) corresponds to data shown in FIG. 2F.

FIG. 3: The Townes mouse model carries human a-globin genes (HBA1 and HBA2) replacing the endogenous mouse a-globin. Because of this, Townes mouse HSCs can be edited using the same reagents for targeting into the human HBA1 locus. The PAH^{enu2} PKU mouse model carries a chemically-induced missense (F263S) mutation in PAH that results in loss of protein activity and lack of response to the BH4 cofactor. The PKU phenotype of homozygous mutant PAH^{enu2/enu2} mice can be corrected by transplant of Townes mouse HSCs that have been edited with either the HBA1-PAH or SFFV-PAH-T2A-YFP vector. Bone marrow is harvested from Townes mice and enriched for CKIT⁺ cells by MACS. Cells are cultured in HSC media at 5% O₂ and expanded over 2 weeks. After 2 weeks, HSPCs are analyzed by FACS for HSC and lineage cell surface markers and edited using the same strategy described in FIGS. 1A-1D and FIGS. 2A-2G. After an additional week in culture, cells are again analyzed by FACS for both YFP % (for the SFFV-PAH-T2A-YFP condition) and HSC or lineage cell surface markers. Cells are then counted and transplanted by retro-orbital injection into 8-12 wk old irradiated PAH^{enu2/enu2} mice. Blood is sampled periodically throughout the experiment for analysis. Bone marrow is harvested at the endpoint.

FIG. 4: PAH requires high levels of its cofactor, BH4, to function. Endogenous levels of BH4 in erythrocytes is below the level required for PAH cofactor activity. Sapropterin is an FDA approved oral BH4 supplement used to treat patients with BH4-responsive forms of PKU. Both phenylalanine and BH4 are soluble across red blood cell membranes; we predict that daily, oral supplementation of sapropterin after transplant will lead to a reduction in plasma phenylalanine levels by PAH activity in circulating edited RBCs. Importantly, the PAH mutation found in the PAH^{enu2} mouse model is unresponsive to BH4. Thus, BH4 supplementation alone will not be sufficient to lower phenylalanine (Phe) levels in the negative control group. Experimental groups: Neg CTL (transplant of unedited Mock HSCs); HBA1-PAH; SFFV-PAH-T2A-YFP. BH4 supplementation begins at 4 weeks post-transplant. Prior to supplementation, blood is drawn for baseline plasma Phe quantification (by Mass/Spec) and peripheral blood analysis (CBC, % YFP+ cells by FACS in the YFP condition). Water is changed daily. 2 weeks after starting BH4 supplementation and every 4 weeks thereafter, peripheral blood (PB) and plasma analysis is repeated. At the endpoint (week 16), in addition to the standard analyses, bone marrow is also harvested and engraftment and targeting rates is determined by FACS and ddPCR.

FIGS. 5A-5B: In vivo PAH data from the experiment illustrated by FIGS. 3 and 4. Homozygous PAH^{enu2/enu2} mice were irradiated at 8 wks old with 8Gray Total Body Irradiation (TBI). Townes mouse HSCs unedited (Mock), edited with HBA1-PAH (HBA1-PAH), or edited with SFFV-PAH-YFP (SFFV-PAH-YFP) were transplanted via Retro-orbital injection the same day. Blood was drawn via retro-orbital bleeds at week 4, 6, and 8. Plasma was separated from whole blood by centrifugation and the plasma [Phe](uM) was determined by LC/MS. FIG. 5A shows plasma Phe concentration data from individual mice across weeks 4-8. BH4 cofactor supplementation in drinking water (20 mg/kg/day dosing) was initiated 1-day after the wk4 samples were obtained. The column on the right of the table shows the average [Phe]ₚₗₐₛₘₐ for wks 6 and 8 for individual mice. FIG. 5B shows graphical representation plasma Phe from the last column of FIG. 5A. Transplant of edited mHSCs expressing PAH driven either by HBA1 (RBC-specific) or SFFV (in all HSC lineages) leads to a significant reduction in [Phe]_{Plasma} in mice with a severe PKU phenotype, compared to controls.

FIG. 6 depicts a schematic of the targeting vector and approach. To engineer RBC-specific expression of coagulation factor IX and to achieve high levels of FIX activity, a vector was designed that facilitates targeted replacement of HBA1 with a hyperactive Factor IX transgene. The Padua variant is a naturally occurring hyperactive variant of Factor IX with a single amino acid substitution (R338L). The targeting vector contains a Padua minigene of codon optimized cDNA+a partially truncated intron 1 (1438 bp long), flanked by Homology Arms (HA) for HBA1: Left HA=466 bp including the 5′UTR of HBA1; right HA=400 bp including 98 bp of the 3′UTR of HBA1.

FIG. 7A: Signal Peptide Background and Optimization: Exon 1 of Factor IX is entirely made up of the 88 bp native signal peptide (SP). Cleavage occurs between the 28^{th} (cysteine) and 29^{th} (threonine) amino acids during post-translational processing. In its native context, the IL6 SP is cleaved between the 27^{th} (proline) and 28th (alanine) amino acids. The Padua construct was redesigned by replacing the first 84 bp of the factor IX signal peptide with the 87 bp IL-6 SP, preserving the original reading frame and leaving the FIX proprotein (formed after cleavage of the signal peptide) intact. In silico prediction of the SP cleavage site for the IL6-Padua recombinant protein was run using SignalP 5.0. The likelihood that a signal peptide exists within the IL6-Padua recombinant protein was 96.06%, and there was a high predicted probability that cleavage would occur at the known cleavage site within the IL6 SP (71.34% probability that the site of cleavage is between the 27^{th} and 28^{th} amino acids).

FIGS. 7B-7D: To compare the two signal peptides, CD34⁺ HSPCs were targeted as described herein with Cas9/sgRNA and an AAV6 carrying either SFFV-Padua or SFFV-I16-Padua. Addition of the SFFV promoter allows all of the optimization experiments to be performed over short term (7 day) HSC cultures, rather than over the course of a 2-week RBC differentiation. After targeting (as described herein), cells were then cultured in HSC media for 7 days with a media change at day 3 and day 6. On day 7, 24 h after the day 6 media change, samples were centrifuged and the cell culture media (supernatant) and whole cell pellet were harvested for analysis. FIG. 7B: Constitutive expression vectors for HSC targeting experiments: SFFV-Padua: The vector described in FIG. 6 was modified by addition of the constitutive SFFV promoter immediately upstream of the start codon for Padua. The transgene is flanked by a 400 bp LHA (66 bp of the original L homology arm was removed, comprising the HBA1 5′UTR) and a 400 bp RHA (unchanged). SFFV-IL6-Padua: As described in FIG. 7A, the first 84 bp of Padua exon 1 was replaced with the 87 bp 1L6-signal peptide. Immediately upstream of this the SFFV promoter was inserted. FIGS. 7C-7D: The amount of factor IX protein contained in the culture supernatants and cell lysates was quantified with a factor IX ELISA. The protein concentrations in the supernatant and lysate were normalized by % targeted alleles, to account for variability in targeting efficiency across different donor HSCs (biological replicates). Bar sections depict the proportion of secreted vs. cytoplasmic Factor IX. This normalization allows for direct (intra-sample) comparison of protein concentration in the secreted vs cellular fractions (FIG. 7C). Secretion efficiency was calculated by dividing the quantity of Factor IX in the secreted fraction by the total (secreted and cellular) quantity (FIG. 7D). The IL-6 signal peptide significantly enhanced secretion by 1.58-fold over the native Factor IX signal peptide (mean secretion efficiency 77.93% and 49.30%, respectively; p<0.001).

FIGS. 8A-8C: HBA1-Padua includes a 1438 bp portion of intron 1, with the total construct being about 4 kb in length. Truncating the intron would reduce the overall construct size and we tested whether it may have a positive impact on targeting rates. New vectors were designed, SFFV-Padua(t) and SFFV-IL6-Padua(t), with a further 139 bp truncation of intron 1 at the two Sca1 restriction enzyme sites in the intron, leaving only a 299 bp portion between exons 1 and 2. FIG. 8A: Vectors with further intron truncation: The (t) denotes a truncated intron. FIG. 8B: Factor IX specific activity, day 7 of HSC culture (24 h in fresh media): Factor IX concentration in the media was quantified by ELISA. Protein activity was measured with a modified aPTT assay, mixing conditioned culture media with factor IX deficient plasma and then measuring clotting activity on a coagulation analyzer. Specific activity was calculated as Units of factor IX activity per mg protein. All cells receiving FIX transgenes (SFFV-Padua, SFFV-Pad(t), SFFV-IL6-Padua, and SFFV-IL6-Pad(t)) showed high FIX specific activities, while no significant differences in specific activity were observed between truncated vs. original-length intron constructs (SFFV-Padua vs. SFFV-Pad(t), or SFFV-IL6-Padua vs. SFFV-IL6-Pad(t)) (paired t-tests, two-tailed, p>0.05). FIG. 8C: Allelic targeting analysis by ddPCR indicates no significant differences in targeting rates between any conditions (paired T-tests, two tailed, p>0.05).

FIGS. 9A-9D: FIX Variants in HSC experiments. FIG. 9A depicts FIX hyperactive variant vectors and the specific amino acid substitutions in each construct. FIG. 9B: Specific activity of factor IX variants: Experiments were performed in HSPCs. Media was changed at day 3 and day 6. 24 h after the day 6 media change, the culture media and cell lysate were collected and analyzed for protein expression and activity. Both the CB2679d mutant (SFFV-IL6-CB267(t), 711.3 U/mg (p<0.001)) and the FIX Mixed variant (SFFV-IL6-Mix(t), 675.7 U/mg (p<0.001)) showed significantly greater specific activity compared to the Padua mutant (SFFV-IL6-pad(t), 263.9 U/mg). There is no significant difference between the CB2679d mutant and the FIX Mixed variant. FIG. 9C: Units of activity/billion targeted alleles: Factor IX activity was normalized by allelic targeting rate and compared to the SFFV-IL6-Padua vector. There is no significant difference between SFFV-IL6-Pad and SFFV-IL6-Pad(t). The CB2679d mutant and the FIX Mixed variant led to 2.698- and 2.474-fold increase, respectively, in factor IX activity over the Padua mutant when normalized by targeting rate. There is no significant difference in FIX activity between the CB2679d mutant and the FIX Mixed variant. FIG. 9D: % Targeted alleles for FIX variant HSC experiments. The integrations of the transgenes SFFV-IL6-CB267(t) and SFFV-IL6-Mix(t) in HSCs reached 29.47% and 28.7% targeted alleles respectively, which were significantly higher than the integration of the transgene SFFV-IL6-Pad(t) (19.38% targeted alleles) (paired T-tests, two tailed, p=0.008 and p=0.01, respectively).

FIGS. 10A-10E: FIX-Variant experiments in RBCs. FIG. 10A depicts an experimental design: human CD34+ HSPCs were targeted with a combined AAV6/Cas9 strategy, cultured for 2 days in HSC media, and then differentiated down the erythroid lineage with a 3-phase, 14-day differentiation protocol. On day 14 cells were counted, spun down, washed with PBS and then replated at ˜3⁶ cells/mL in modified Phase 3 RBC media (lacking human serum and plasma). 24 hours later the cell culture supernatant was collected to measure Factor IX activity using a modified aPTT assay. FIG. 10B depicts schematic drawings of RBC-specific IL6-FIX variant constructs driven by the endogenous HBA1 promoter: Padua (control vector), IL6-CB2679d (truncated intron), and IL6-Mixed Variant (truncated intron). FIG. 10C: Allelic targeting analysis by ddPCR on samples taken at day 14 of the RBC differentiation. There is no significant difference between the groups HBA1-Padua (21.6%), HBA1-IL6-CB2679(t) (27.56%), and HBA1-1L6-Mix(t) (26.38%) respectively (p>0.05 for all comparisons). FIG. 10D depicts RBC differentiation analysis by FACS on day 14. There is no significant difference between the groups HBA1-Padua, HBA1-IL6-CB2679(t), and HBA1-IL6-Mix(t). FIG. 10E depicts FIX activity by measuring U/10⁹ targeted alleles of RBC sampled from d15 cell culture supernatant. Both the HBA1-1L6-CB2679(t) and HBA1-IL6-Mix groups showed significant increase in factor IX activity, leading to a 2.586- and 2.079-fold higher, respectively, over HBA1-Padua.

FIG. 11: Exemplary hemophilia B mouse experiments: The B6.129P2-F9^{tm1Dws}/J mouse model (factor IX knockout) is used for these experiments. Townes HSCs are harvested, expanded, edited and transplanted as described for the PKU mouse experiments. The radiation dose can be titrated to assess if disease correction can be achieved with half or low dose (or potentially no) radiation.

FIGS. 12A-12D depict tEPOR Enrichment of HBA1-Factor IX targeted RBCs. FIG. 12A depicts the experimental design. Briefly, human CD34+ HSPCs were targeted with a combined AAV6/Cas9 strategy, cultured for 2 days in HSC media, and then differentiated down the erythroid lineage with a 3-phase, 14-day differentiation protocol. On day 14 cells were counted, spun down, washed with PBS and then replated at ˜3⁶ cells/mL in modified Phase 3 RBC media (lacking human serum and plasma). 24 hours later the cell culture supernatant was collected to measure Factor IX activity using a modified aPTT assay. FIG. 12B depicts vector design: The first vector HBA1-IL6-CB2679(t) was the same vector from the earlier FIX Variant RBC experiments (FIG. 10B). The second vector HBA1-IL6-CB2679(t)-T2A-tEPOR is a modification of the first vector by including a T2A sequence immediately downstream of the CB2679 FIX variant gene (but prior to the stop codon), followed by a truncated erythropoietin receptor (tEPOR) gene sequence. FIG. 12C depicts allelic targeting analysis and tEPOR enrichment by ddPCR IL6-CB2679-tEPOR targeted alleles enriched 1.7-fold from day 0 (33.1%) to day 14 (55.2%), and IL6-CB2679-tEPOR had 1.4× higher targeting than IL6-CB2679 on d14 (55.2% vs. 40.3%). FIG. 12D depicts Factor IX Activity (U/10⁹ cells/mL) using a modified aPTT coagulation assay with day 15 cell culture media. IL6-CB2679-tEPOR had 1.2-fold greater Factor IX activity than IL6-CB2679 (3.139 vs. 2.716 U/10⁹ cells/mL).

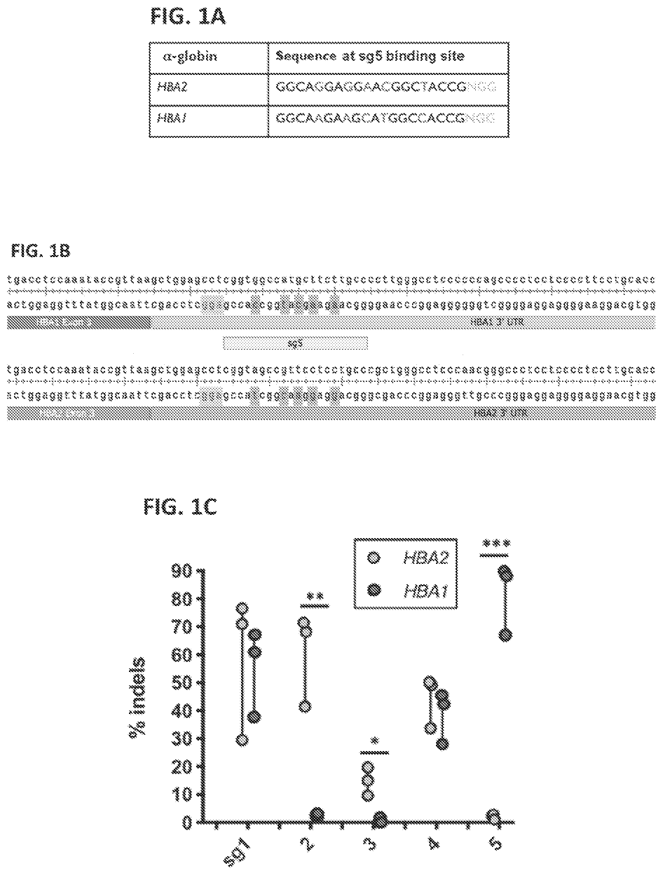

### DETAILED DESCRIPTION

### 1. Introduction

The present disclosure provides methods and compositions for integrating transgenes, e.g., for therapeutic genes such as FIX or PAH, into the HBA1 or HBA2 locus in hematopoietic stem and progenitor cells (HSPCs). The present methods can be used to introduce transgenes, e.g., coding sequences with optional elements such as promoters or other regulatory elements (e.g., enhancers, repressor domains), introns, WPREs, poly A regions, UTRs (e.g., 3′ UTRs), specifically into the HBA1 or HBA2 locus of HSPCs. The guide RNAs used in the present methods specifically recognize HBA1 but not HBA2, or HBA2 but not HBA1, enabling the selective cleavage of either HBA1 or HBA2 by an RNA-directed nuclease such as Cas9. By cleaving HBA1 or HBA2, but not both, in the presence of a donor template comprising a transgene, the transgene can integrate into the genome at the site of cleavage by homology directed recombination (HDR), e.g., replacing the endogenous HBA1 or HBA2 gene.

The present disclosure provides methods and compositions for gene therapy for genetic diseases, including hemophilia B and phenylketonuria (PKU), by engineering erythroid-specific expression of factor IX, and phenylalanine hydroxylase (PAH), respectively. Due to the vast quantities of erythrocytes produced daily and their whole-body distribution, as well as the robust levels of erythroid-specific expression achieved due to the strength and specificity of the endogenous HBA1 (or HBA2) promoter, the present methods allow the use of red blood cells as protein factories that deliver therapeutic payloads of the disease-correcting proteins throughout the body. The present disclosure provides novel methods and compositions by which transgenes have been engineered to optimize expression, activity, and secretion of the therapeutic protein, allowing the production of sufficient levels of protein activity to ameliorate the disease phenotype with even sub-standard amounts of bone marrow conditioning before transplant.

### 2. General

Practicing this invention utilizes routine techniques in the field of molecular biology. Basic texts disclosing the general methods of use in this invention include Sambrook and Russell, Molecular Cloning, A Laboratory Manual (3rd ed. 2001). Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a PAH or FIX cDNA, transgene, or encoded protein. In some embodiments, the term refers to the production of a transcriptional and/or translational product encoded by a gene or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215: 403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V. and VI as well as many sub-types, with Class 1 including types I and 111 CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “homologous repair template” refers to a polynucleotide sequence that can be used to repair a double-stranded break (DSB) in the DNA, e.g., a CRISPR/Cas9-mediated break at the HBA1 or HBA2 locus as induced using the herein-described methods and compositions. The homologous repair template comprises homology to the genomic sequence surrounding the DSB, i.e., comprising HBA1 or HBA2 homology arms. In some embodiments, two distinct homologous regions are present on the template, with each region comprising at least 50, 100, 200, 300, 400, 500, 600, 700, 800, 900 or more nucleotides or more of homology with the corresponding genomic sequence. In particular embodiments, the templates comprise two homology arms comprising about 500 nucleotides of homology extending from either site of the sgRNA target site. The repair template can be present in any form, e.g., on a plasmid that is introduced into the cell, as a free floating doubled-stranded DNA template (e.g., a template that is liberated from a plasmid in the cell), or as single-stranded DNA. In particular embodiments of the present disclosure, the template is present within a viral vector, e.g., an adeno-associated viral vector such as AAV6. The templates of the present disclosure can also comprise a transgene, e.g., PAH or FIX transgene.

“HBA1” and “HBA2” (hemoglobin subunit alpha 1 and 2, respectively) are closely related, but not identical, genes encoding alpha-globin, which is a component of hemoglobin. HBA1 and HBA2 are located within the alpha-globin locus, located on human chromosome 16. Their coding sequences are identical, but the genes diverge, e.g., in the 5′UTRs, introns, and particularly the 3′UTRs. The NCBI gene ID for HBA1 is 3039, and the NCBI gene ID for HBA2 is 3040, the entire disclosures of which are herein incorporated by reference.

PAH (phenylalanine hydroxylase) is a gene encoding the phenylalanine hydroxylase enzyme, which converts phenylalanine to tyrosine and which is the rate-limiting step in phenylalanine catabolism. Homozygous PAH mutations that lead to PAH deficiencies can cause phenylketonuria (PKU), characterized by high levels of phenylalanine, which if left untreated can result in irreversible neurologic damage. The NCBI gene ID for human PAH is 5053, and the UniProt ID is P00439, the entire disclosures of which are herein incorporated by reference. It will be appreciated that the present methods can be used to treat PKU resulting from any mutation in the PAH gene, e.g., missense or nonsense mutations, deletions, etc.

FIX (factor IX) is a gene encoding a coagulation factor that is expressed as a zymogen and then converted to an active form by factor XIa (of the contact pathway) or factor VIIa (of the tissue factor pathway) to produce a two-chain form, where the chains are linked by a disulfide bridge. Deficiency of this protein causes hemophilia B. The NCBI gene ID for human FIX is 2158, and the UniProt ID is P00740, the entire disclosures of which are herein incorporated by reference. It will be appreciated that the present methods can be used to treat hemophilia B resulting from any mutation in the FIX gene, e.g., missense or nonsense mutations, deletions, etc.

EPOR (erythropoietin receptor) is the receptor for erythropoietin (EPO), a cytokine that regulates the proliferation and differentiation of erythroid precursor cells. When italicized (i.e., EPOR), EPOR refers to a polynucleotide (e.g., gene, locus, transgene, coding sequence, cDNA, expression cassette) encoding EPOR Upon binding of EPO, EPOR activates JAK2 tyrosine kinase, which in turn activates different intracellular pathways such as Ras/MAP kinase, PI3 kinase, and STAT transcription factors. EPOR is a member of the cytokine receptor family, and the EPOR gene is located on human chromosome 19p (19p13.2). The NCBI gene ID for human EPOR is 2057, and the UniProt ID for human EPOR is P19235, the entire disclosures of which are herein incorporated by reference.

Truncated EPOR, or tEPOR (encoded by tEPOR), refers to forms of the EPO receptor, or to polynucleotides encoding the receptor forms, that lack a portion or all of the receptor's cytoplasmic domain. For example, in some embodiments a tEPOR lacks the 70 C-terminal amino acids of full-length EPOR. In some embodiments, a tEPOR lacks all 236 amino acids of the cytoplasmic domain. In some embodiments, a tEPOR lacks, e.g., about 10, 20, 30, 40, 50, 60, 70, 80, 90, 100, 110, 120, 130, 140, 150, 160, 170, 180, 190, 200, 210, 220, 230, 10-236, 10-50, 50-60, 60-70, 65-75, 70-80, 80-90, 90-100, 100-150, 150-200, or 200-236 amino acids. In some embodiments, a tEPOR lacks a binding site and/or does not interact with the tyrosine phosphatase SHP-1 (or SHPTP-1), which normally plays a role in inhibiting EPOR signaling. In some embodiments, a coding sequence (e.g., gene or transgene) encoding a tEPOR comprises a nonsense mutation in exon 7 or exon 8, and/or encodes any of the herein-described forms of truncated EPOR. Nonsense mutations causing the expression of truncated EPOR act as dominant mutations that render cells hypersensitive to EPO, leading to an ability to undergo effective proliferation and differentiation in the presence of reduced amounts of EPO, and to show enhanced levels of proliferation and differentiation in the presence of normal EPO levels.

As used herein, “homologous recombination” or “HR” refers to insertion of a nucleotide sequence during repair of double-stranded breaks in DNA via homology-directed repair mechanisms. This process uses a “donor template” or “homologous repair template” with homology to nucleotide sequence in the region of the break as a template for repairing a double-stranded break. The presence of a double-stranded break facilitates integration of the donor sequence. The donor sequence may be physically integrated or used as a template for repair of the break via homologous recombination, resulting in the introduction of all or part of the nucleotide sequence. This process is used by a number of different gene editing platforms that create the double-stranded break, such as meganucleases, zinc finger nucleases (ZFNs), transcription activator-like effector nucleases (TALENs), and the CRISPR-Cas9 gene editing systems. In particular embodiments of the present disclosure, HR involves double-stranded breaks induced by CRISPR-Cas9.

### 4. CRISPR/Cas Systems Specifically Targeting the HBA1 or HBA2 Locus

The present disclosure is based in part on the identification of CRISPR guide sequences that specifically direct the cleavage of HBA1 or HBA2 by RNA-guided nucleases but without leading to cleavage of both genes. The present disclosure provides a CRISPR/AAV6-mediated genome editing method that can achieve high rates of targeted integration at both loci. The integrated transgenes exhibit RBC-specific expression of functional transgenes, and cells edited at this locus are capable of long-term engraftment and hematopoietic reconstitution.

Because of the redundancy of HBA1 and HBA2, integration at this locus allows delivery of transgenes for RBC-specific expression without the risk of bi-allelic integrations causing detrimental cellular effects.

sgRNAs

The single guide RNAs (sgRNAs) used in the present disclosure target either the HBA1 or HBA2 locus, but not both. sgRNAs interact with a site-directed nuclease such as Cas9 and specifically bind to or hybridize to a target nucleic acid within the genome of a cell, such that the sgRNA and the site-directed nuclease co-localize to the target nucleic acid in the genome of the cell. The sgRNAs as used herein comprise a targeting sequence comprising homology (or complementarity) to a target DNA sequence at the HBA1 or HBA2 locus, and a constant region that mediates binding to Cas9 or another RNA-guided nuclease. The sgRNA can target any sequence within HBA1 or HBA2 adjacent to a PAM sequence. In particular embodiments, the sgRNA targets HBA1 but not HBA2, or HBA2 but not HBA1, i.e., the sgRNA can target any sequence within the HBA1 or HBA2 genes that are distinct between the two genes and adjacent to a PAM sequence. In particular embodiments, a single guide RNA, or sgRNA, is used. In some embodiments, the target sequence is within intron 2 or the 3′ UTR of HBA1 or HBA2. In particular embodiments, the target sequence is within the 3′ UTR. In particular embodiments, the target sequence differs by 3, 4, 5 or more nucleotides between HBA1 and HBA2. In some embodiments, the target sequence comprises one of the sequences shown as SEQ ID NO:1 or 2, or a sequence comprising 1, 2, 3 or more mismatches with SEQ ID NO:1 or 2. In particular embodiments, the target sequence comprises the target sequence of sg5 (SEQ ID NO:1) or sg2 (SEQ ID NO:2).

In some embodiments, the sgRNAs comprise one or more modified nucleotides. For example, the polynucleotide sequences of the sgRNAs may also comprise RNA analogs, derivatives, or combinations thereof. For example, the probes can be modified at the base moiety, at the sugar moiety, or at the phosphate backbone (e.g., phosphorothioates). In some embodiments, the sgRNAs comprise 3′ phosphorothiate internucleotide linkages, 2′-O-methyl-3′-phosphoacetate modifications, 2′-fluoro-pyrimidines, S-constrained ethyl sugar modifications, or others, at one or more nucleotides. In particular embodiments, the sgRNAs comprise 2′-O-methyl-3′-phosphorothioate (MS) modifications at one or more nucleotides (see, e.g., Hendel et al. (2015) Nat. Biotech. 33(9):985-989, the entire disclosure of which is herein incorporated by reference). In particular embodiments, the 2′-O-methyl-3′-phosphorothioate (MS) modifications are at the three terminal nucleotides of the 5′ and 3′ ends of the sgRNA.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### RNA-Guided Nucleases

Any CRISPR-Cas nuclease can be used in the method, i.e., a CRISPR-Cas nuclease capable of interacting with a guide RNA and cleaving the DNA at the target site as defined by the guide RNA. In some embodiments, the nuclease is Cas9 or Cpf1. In particular embodiments, the nuclease is Cas9. The Cas9 or other nuclease used in the present methods can be from any source, so long that it is capable of binding to an sgRNA of the disclosure and being guided to and cleaving the specific HBA1 or HBA2 sequence targeted by the targeting sequence of the sgRNA. In particular embodiments, the Cas9 is from Streptococcus pyogenes. In some embodiments, the Cas9 is a high fidelity Cas9.

Also disclosed herein are CRISPR/Cas or CRISPR/Cpf1 systems that target and cleave DNA at the HBA1 or HBA2 locus. An exemplary CRISPR/Cas system comprises (a) a Cas (e.g., Cas9) or Cpf1 polypeptide or a nucleic acid encoding said polypeptide, and (b) an sgRNA that hybridizes specifically to HBA1 or HBA2, or a nucleic acid encoding said guide RNA. In some instances, the nuclease systems described herein, further comprises a donor template comprising a FIX or PAH or tEPOR transgene as described herein. In certain embodiments, the donor template is a bicistronic cassette encoding two transgenes, (e.g., a FIX-tEPOR fusion protein). In certain embodiments, the donor template is a bicistronic cassette comprising a nucleic acid sequence encoding a 2A cleavage peptide (e.g., a member of the 2A peptide family such as a T2A, P2A, E2A, or F2A cleavage peptide) between the two transgenes (e.g., FIX-T2A-tEPOR). In particular embodiments, the CRISPR/Cas system comprises an RNP comprising an sgRNA targeting HBA1 or HBA2 and a Cas protein such as Cas9.

In addition to the CRISPR/Cas9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used to induce a single or double stranded break at the HBA1 or HBA2 locus to carry out the methods disclosed herein.

Introducing the sgRNA and Cas Protein into Cells

The guide RNA and nuclease can be introduced into the cell using any suitable method, e.g., by introducing one or more polynucleotides encoding the guide RNA and the nuclease into the cell, e.g., using a vector such as a viral vector or delivered as naked DNA or RNA, such that the guide RNA and nuclease are expressed in the cell. In particular embodiments, the guide RNA and nuclease are assembled into ribonucleoproteins (RNPs) prior to delivery to the cells, and the RNPs are introduced into the cell by, e.g., electroporation.

Animal cells, mammalian cells, preferably human cells, modified ex vivo, in vitro, or in vivo are contemplated. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, or a peripheral blood mononuclear cell (PBMC). In particular embodiments, the cells are CD34⁺ hematopoietic stem and progenitor cells (HSPCs), e.g., cord blood-derived (CB), adult peripheral blood-derived (PB), or bone marrow derived HSPCs.

HSPCs can be isolated from a subject, e.g., by collecting mobilized peripheral blood and then enriching the HSPCs using the CD34 marker. In some embodiments, the cells are from a subject with hemophilia B. In some embodiments, the cells are from a subject with phenylketonuria. In some such embodiments, the transgene that is integrated into the genome of the HSPC is PAH or FIX, e.g., at the HBA1 locus. In one embodiment, a method is provided of treating a subject with phenylketonuria, comprising genetically modifying a plurality of HSPCs isolated from the subject so as to integrate the PAH gene at the HBA1 locus, and reintroducing the HSPCs into the subject. In some such embodiments, the subject is further administered BH4. In another embodiment, a method is provided of treating a subject with hemophilia B, comprising genetically modifying a plurality of HSPCs isolated from the subject so as to integrate the FIX gene at the HBA1 locus, and reintroducing the HSPCs into the subject.

To avoid immune rejection of the modified cells when administered to a subject, the cells to be modified are preferably derived from the subject's own cells. Thus, preferably the mammalian cells are autologous cells from the subject to be treated with the modified cells.

In some embodiments, cells are harvested from the subject and modified according to the methods disclosed herein, which can include selecting certain cell types, optionally expanding the cells and optionally culturing the cells, and which can additionally include selecting cells that contain the transgene integrated into the HBA1 or HBA2 locus. In particular embodiments, such modified cells are then reintroduced into the subject.

Further disclosed herein are methods of using said nuclease systems to produce the modified host cells described herein, comprising introducing into the cell (a) an RNP of the disclosure that targets and cleaves DNA at the HBA1 or HBA2 locus, and (b) a homologous donor template or vector as described herein. Each component can be introduced into the cell directly or can be expressed in the cell by introducing a nucleic acid encoding the components of said one or more nuclease systems.

Such methods will target integration of the functional transgene, e.g., PAH or FIX transgene, at the endogenous HBA1 or HBA2 locus in a host cell ex vivo. Such methods can further comprise (a) introducing a donor template or vector into the cell, optionally after expanding said cells, or optionally before expanding said cells, and (b) optionally culturing the cell.

In some embodiments, the disclosure herein contemplates a method of producing a modified mammalian host cell, the method comprising introducing into a mammalian cell: (a) an RNP comprising a Cas nuclease such as Cas9 and an sgRNA specific to the HBA1 or HBA2 locus, and (b) a homologous donor template or vector as described herein.

In any of these methods, the nuclease can produce one or more single stranded breaks within the HBA1 or HBA2 locus, or a double-stranded break within the HBA1 or HBA2 locus. In these methods, the HBA1 or HBA2 locus is modified by homologous recombination with said donor template or vector to result in insertion of the transgene into the locus. The methods can further comprise (c) selecting cells that contain the transgene integrated into the HBA1 or HBA2 locus.

In some embodiments, i53 (Canny et al. (2018) Nat Biotechnol 36:95) is introduced into the cell in order to promote integration of the donor template by homology directed repair (HDR) versus integration by non-homologous end-joining (NHEJ). For example, an i53 polypeptide or an mRNA encoding i53 can be introduced into the cell, e.g., by electroporation at the same time as an sgRNA-Cas9 RNP. The sequence of i53 can be found, inter alia, at www.addgene.org/92170/sequences/.

Techniques for the insertion of transgenes, including large transgenes, capable of expressing functional proteins, including enzymes, cytokines, antibodies, and cell surface receptors are known in the art (See, e.g., Bak and Porteus, Cell Rep. 2017 Jul. 18; 20(3): 750-756 (integration of EGFR); Kanojia et al., Stem Cells. 2015 October; 33(10):2985-94 (expression of anti-Her2 antibody); Eyquem et al., Nature. 2017 Mar. 2; 543(7643):113-117 (site-specific integration of a CAR); O'Connell et al., 2010 PLoS ONE 5(8): e12009 (expression of human IL-7); Tuszynski et al., Nat Med. 2005 May; 11(5):551-5 (expression of NGF in fibroblasts); Sessa et al., Lancet. 2016 Jul. 30; 388(10043):476-87 (expression of arylsulfatase A in ex vivo gene therapy to treat MLD); Rocca et al., Science Translational Medicine 25 Oct. 2017: Vol. 9, Issue 413, eaaj2347 (expression of frataxin); Bak and Porteus, Cell Reports, Vol. 20, Issue 3, 18 Jul. 2017, Pages 750-756 (integrating large transgene cassettes into a single locus), Dever et al., Nature 17 Nov. 2016: 539, 384-389 (adding tNGFR into hematopoietic stem cells (HSC) and HSPCs to select and enrich for modified cells); each of which is herein incorporated by reference in its entirety.

### Homologous Repair Templates

In particular embodiments, the transgene to be integrated, which is comprised by a polynucleotide or donor construct, is a FIX or PAH transgene. Such transgenes can be expressed in red blood cells and serve to replace or compensate for a defective gene, e.g., a defective FIX gene in a subject with hemophilia B, or a defective PAH gene in a subject with PKU. In particular embodiments, the genetically modified HSPCs are introduced into a subject and differentiate into red blood cells, and the red blood cells then circulate and supply the encoded protein in vivo.

The transgene comprises a functional coding sequence for a gene, e.g., a gene that is defective in a subject, with optional elements such as promoters or other regulatory elements (e.g., enhancers, repressor domains), introns, WPREs, poly A regions, UTRs (e.g., 3′ UTRs).

In some embodiments, the transgene in the homologous repair template comprises or is derived from a cDNA for the corresponding gene. In some embodiments, the transgene in the homologous repair template comprises the coding sequence from the corresponding gene and one or more introns. In some embodiments, the transgene in the homologous repair template is codon-optimized, e.g., comprises at least 70%, 75%, 80%, 85%, 90%, 95%, or more homology to the corresponding wild-type coding sequence or cDNA, or a fragment thereof.

In particular embodiments, the template further comprises a polyA sequence or signal, e.g., a bovine growth hormone polyA sequence or a rabbit beta-globin polyA sequence, at the 3′ end of the cDNA. In particular embodiments, a Woodchuck Hepatitis Virus Posttranscriptional Regulatory Element (WPRE) is included within the 3′UTR of the template, e.g., between the 3′ end of the coding sequence and the 5′ end of the polyA sequence, so as to increase the expression of the transgene. Any suitable WPRE sequence can be used; See, e.g., Zufferey et al. (1999) J. Virol. 73(4):2886-2892; Donello, et al. (1998). J Virol 72: 5085-5092; Loeb, et al. (1999). Hum Gene Ther 10: 2295-2305; the entire disclosures of which are herein incorporated by reference).

In particular embodiments, the transgene encodes Factor IX (FIX). In particular embodiments, the (FIX) transgene comprises the nucleotide sequence shown as any one of SEQ ID NOS: 6-11 or a subsequence thereof, or a sequence comprising at least about 709%, 75%, 80%, 85%, 90%, 95%, 96%, 97%, 98%, 99%, or more to any one of SEQ ID NOS: 6-11 or a subsequence thereof. In some embodiments, the FIX transgene is codon optimized.

In particular embodiments, the FIX transgene comprises one or more of the following features relative to a wild-type FXI coding sequence: i) the presence of two or more mutations selected from the group consisting of R318Y, R338E, R338L, and T343R; ii) the inclusion of an exogenous signal sequence, e.g., a signal sequence from an IL-6 protein; and iii) a truncation in one or more introns, e.g., in intron 1.

In particular embodiments, the FIX transgene comprises one or more mutations relative to a wild-type FIX sequence. For example, in some embodiments, the transgene comprises a “Padua” variant mutation at position 338, i.e., an R338L substitution. In some such embodiments, the transgene comprises a nucleotide sequence shown as SEQ ID NOS: 6-8 or a subsequence thereof, or a sequence comprising at least about 70%, 75%, 80%, 85%, 90%, 95%, 96%, 97%, 98%, 99%, or more to one or more of SEQ ID NOS: 6-8 or a subsequence thereof. In some embodiments, the transgene comprises two or more mutations of a “CB 2679d-GT” variant, i.e., R318Y, R338E, and/or T343R mutations. See, e.g., Nair et al., Blood (2021) 137 (21): 2902-2906. In some such embodiments, the transgene comprises the nucleotide sequence shown as SEQ ID NO: 9 or a subsequence thereof, or a sequence comprising at least about 70%, 75%, 80%, 85%, 90%, 95%, 96%, 97%, 98%, 99%, or more to SEQ ID NO: 9 or a subsequence thereof. In particular embodiments, the transgene comprises the mutations R318Y, R338L, and T343R In some such embodiments, the transgene comprises the nucleotide sequence shown as SEQ ID NO: 10 or a subsequence thereof, or a sequence comprising at least about 70%, 75%, 80%, 85%, 90%, 95%, 96%, 97%, 98%, 99%, or more to SEQ ID NO: 10 or a subsequence thereof. In particular embodiments, a mutant FIX protein encoded by the transgene comprises greater activity than a wild-type FIX protein, e.g., an increase in activity of at least about 10%, 20%, 30%, 40%, 50%, 60%, 70%, 80%, 90%, 100%, or more relative to the activity of a wild type FIX enzyme.

In particular embodiments, the Factor IX transgene comprises an exogenous signal peptide, i.e., the native signal peptide at the beginning of exon 1 is replaced with a signal peptide from another source. In particular embodiments, the native signal peptide is replaced with a signal peptide from IL-6, e.g., as in any one of SEQ ID NOS: 6-11. In particular embodiments, the FIX protein expressed from the transgene comprising the exogenous signal peptide is more highly secreted than a wild-type FIX protein, e.g., as secreted from red blood cells. In particular embodiments, the FIX protein is secreted with at least about 3-fold greater efficiency than a wild-type FIX protein.

In some embodiments, the Factor IX transgene comprises a first exon 1, followed by an intron 1, followed by exons 2-8. In particular embodiments, the intron 1 is truncated. For example, the approximately 6.2 kb intron 1 can be truncated by, e.g., at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, or more kb. In particular embodiments, the truncation removes about 4.8 kb from the interior of intron 1, e.g., the nucleotides between two PvuII sites at nucleotides 1098 and 5882. In particular embodiments, the truncation removes about 5.9 kb from the interior of intron 1, e.g., the nucleotides between the two ScaI sites at nucleotides 258 and 6168. In certain embodiments, the Factor IX transgene comprises a portion of intron 1 of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, or 6.1 kb. In particular embodiments, the Factor IX transgene comprises a portion of intron 1 of about 0.3 or about 1.4 kb.

In particular embodiments, the transgene encodes phenylalanine hydroxylase (PAH). In particular embodiments, the PAH transgene is codon optimized. In particular embodiments, the PAH transgene comprises the nucleotide sequence shown as SEQ ID NO: 5 or a subsequence thereof, or a sequence comprising at least about 70%, 75%, 80%, 85%, 90%, 95%, 96%, 97%, 98%, 99%, or more to SEQ ID NO: 5 or a subsequence thereof.

In particular embodiments, the transgene encodes a truncated EPO receptor (tEPOR). In particular embodiments, the tEPOR transgene is in fusion with the FIX or PAH transgene. In certain embodiments, the donor template is a bicistronic cassette comprising two transgenes (e.g., FIX and tEPOR). In certain embodiments, the donor template is a bicistronic cassette comprising a nucleic acid sequence encoding a 2A cleavage peptide (e.g., a member of the 2A peptide family such as a T2A, P2A, E2A, or F2A cleavage peptide) between the two transgenes (e.g., FIX-T2A-tEPOR). An exemplary nucleic acid sequence encoding a fusion protein linked by a T2A cleavage peptide, FIX-T2A-tEPOR, is shown as SEQ ID NO: 11. In some instances, the 2A cleavage peptide is a T2A or P2A cleavage peptide. In other instances, the 2A cleavage peptide is a peptide having sequence similarity and functional interchangeability to a T2A or P2A cleavage peptide, such as an E2A or F2A cleavage peptide.

To facilitate homologous recombination, the transgene is flanked within the polynucleotide or donor construct by sequences homologous to the target genomic sequence. For example, the transgene can be flanked by sequences surrounding the site of cleavage as defined by the guide RNA. In particular embodiments, the transgene is flanked by sequences homologous to the 3′ and to the 5′ ends of the HBA1 or HBA2 gene or coding sequence, such that the HBA1 or HBA2 gene is replaced upon the HDR-mediated integration of the transgene. In one such embodiment, the transgene is flanked on one side by a sequence corresponding to the 3′ UTR of the HBA1 or HBA2 gene, and on the other side by a sequence corresponding to the region of the transcription start site, e.g., just 5′ of the start site, of HBA1 or HBA2. The homology regions can be of any size, e.g., 100-1000 bp, 300-800 bp, 400-600 bp, or about 100, 200, 300, 400, 500, 600, 700, 800, 900, 1000, or more bp. In some embodiments, the transgene comprises a promoter, e.g., a constitutive or inducible promoter, such that the promoter drives the expression of the transgene in vivo. In particular embodiments, the transgene replaces the coding sequence of HBA1 or HBA2 such that its expression is driven by the endogenous HBA1 or HBA2 promoter. In particular embodiments, the donor template comprises a sequence comprising at least about 70%, 75%, 80%, 85%, 90%, 95%, 96%, 97%, 98%, 99%, or more identity to SEQ ID NO:3, or a fragment thereof. In particular embodiments, the donor template comprises the sequence shown as SEQ ID NO:4, or a fragment thereof.

Any suitable method can be used to introduce the polynucleotide, or donor construct, into the cell. In some instances, the donor template is single stranded, double stranded, a plasmid or a DNA fragment. In some instances, plasmids comprise elements necessary for replication, including a promoter and optionally a 3′ UTR. The vector can be a viral vector, such as a retroviral, lentiviral (both integration competent and integration defective lentiviral vectors), adenoviral, adeno-associated viral or herpes simplex viral vector. Viral vectors may further comprise genes necessary for replication of the viral vector. In particular embodiments, the polynucleotide is introduced using a recombinant adeno-associated viral vector, e.g., rAAV6.

In some embodiments, the targeting construct comprises: (1) a viral vector backbone, e.g. an AAV backbone, to generate virus; (2) arms of homology to the target site of at least 200 bp but ideally at least 400 bp on each side to assure high levels of reproducible targeting to the site (see, Porteus, Annual Review of Pharmacology and Toxicology, Vol. 56:163-190 (2016); which is hereby incorporated by reference in its entirety); (3) a transgene encoding a functional protein and capable of expressing the functional protein, a polyA sequence, and optionally a WPRE element; and optionally (4) an additional marker gene to allow for enrichment and/or monitoring of the modified host cells. Any AAV known in the art can be used. In some embodiments the primary AAV serotype is AAV6. In some embodiments, the vector, e.g., rAAV6 vector, comprising the donor template is from about 1-2 kb, 2-3 kb, 3-4 kb, 4-5 kb, 5-6 kb, 6-7 kb, 7-8 kb, or larger.

Suitable marker genes are known in the art and include Myc, HA, FLAG, GFP, truncated NGFR, truncated EGFR, truncated CD20, truncated CD19, as well as antibiotic resistance genes. In some embodiments, the homologous repair template and/or vector (e.g., AAV6) comprises an expression cassette comprising a coding sequence for truncated nerve growth factor receptor (tNGFR), operably linked to a promoter such as the Ubiquitin C promoter.

In some embodiments, the donor template or vector comprises a nucleotide sequence homologous to a fragment of the HBA1 or HBA2 locus, or a nucleotide sequence is at least 85%, 88%, 90%, 926, 95%, 98%, or 99% identical to at least 200, 250, 300, 350, 400, 450, 500, or more consecutive nucleotides of the HBA1 or HBA2 locus.

The inserted construct can also include other safety switches, such as a standard suicide gene into the locus (e.g., iCasp9) in circumstances where rapid removal of cells might be required due to acute toxicity. The present disclosure provides a robust safety switch so that any engineered cell transplanted into a body can be eliminated, e.g., by removal of an auxotrophic factor. This is especially important if the engineered cell has transformed into a cancerous cell.

The present methods allow for the efficient integration of the donor template at the endogenous HBA1 or HBA2 locus. In some embodiments, the present methods allow for the insertion of the donor template in about 10%, 15%, 20%, 25%, 30%, 35%, 40%, or more cells, e.g., cells from an individual with hemophilia B or phenylketonuria. The methods also allow for high levels of expression of the encoded protein in cells, e.g., cells from an individual with hemophilia B or phenylketonuria, with an integrated transgene, e.g., levels of expression that are at least about 70%, 75%, 80%, 85%, 90%, 95%, or more relative to the expression in healthy control cells.

### 5. Methods of Treatment

Following the integration of the transgene into the genome of the HSPC and confirming expression of the encoded therapeutic protein, a plurality of modified HSPCs can be reintroduced into the subject. In one embodiment, the HSPCs are introduced by intrafemoral injection, such that they can populate the bone marrow and differentiate into, e.g., red blood cells. In some embodiments, the HSPCs are induced to differentiate into red blood cells in vitro, and the modified red blood cells are then re-introduced into the subject.

Disclosed herein, in some embodiments, are methods of treating a genetic disorder, e.g., phenylketonuria or hemophilia B in an individual in need thereof, the method comprising providing to the individual a protein replacement therapy using the genome modification methods disclosed herein. In some instances, the method comprises a modified host cell ex vivo, comprising a functional transgene, e.g., PAH or FIX transgene, integrated at the HBA1 or HBA2 locus, wherein the modified host cell expresses the encoded protein which is deficient in the individual, thereby treating the genetic disorder in the individual.

In some embodiments, HSPCs from an individual with phenylketonuria are modified to introduce the herein-described PAH transgene, and are subsequently reintroduced into the individual. In some embodiments, the individual is further administered tetrahydrobiopterin (BH4), e.g., by oral administration.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals.

In some embodiments, a pharmaceutical composition comprising a modified autologous host cell of the disclosure is provided. The modified autologous host cell is genetically engineered to comprise an integrated transgene at the HBA1 or HBA2 locus. The modified host cell of the disclosure herein may be formulated using one or more excipients to, e.g.: (1) increase stability; (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g., the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regime before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing the disorder, e.g., hemophilia B or phenylketonuria. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more modified cells to the subject, or any amount sufficient to obtain the desired therapeutic or prophylactic, effect. The desired dosage of the modified host cells of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years or more than 10 years.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of hemophilia B or phenylketonuria or other genetic disorder is also encompassed by the disclosure.

The present disclosure also contemplates kits comprising compositions or components described herein, e.g., sgRNA, Cas9, RNPs, i53, and/or homologous templates comprising PAH and/or FIX transgenes, as well as, optionally, reagents for, e.g., the introduction of the components into cells. The kits can also comprise one or more containers or vials, as well as instructions for using the compositions in order to modify cells and treat subjects according to the methods described herein.

### 6. Examples

### Example 1. Targeted Integration of Codon-Optimized cDNA of PAH into HBA1 Locus

In this study, we have leveraged the combined Cas9/AAV6 genome editing method to mediate site-specific integration of a PAH cDNA transgene into the HBA1 locus while leaving the virtually identical HBA2 gene unperturbed. We found that this process allowed us to replace the entire coding region of HBA1 with PAH transgene at high frequencies, which allowed high levels of PAH expression in differentiated RBCs.

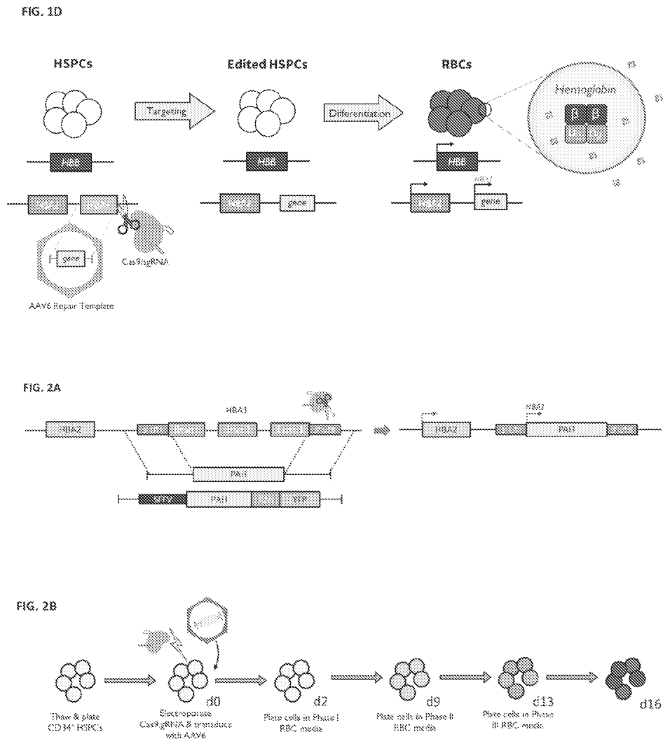

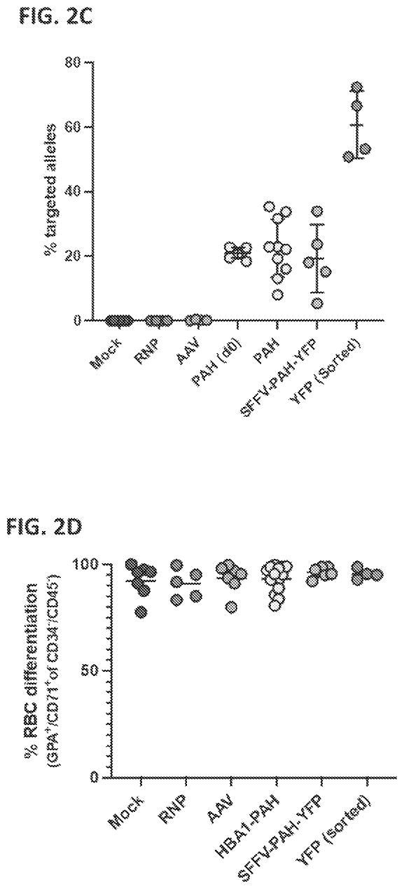

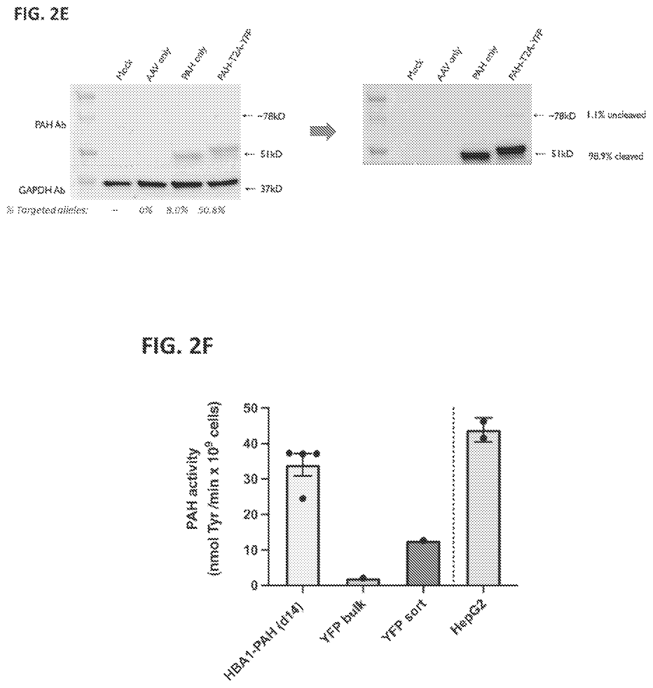

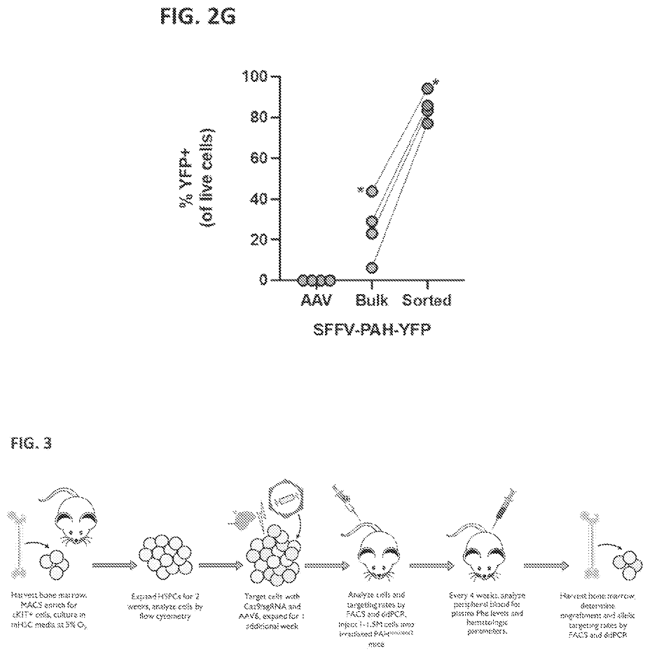

We generated a codon-optimized PAH transgene and integrated it into a donor template where it was flanked by two HBA1 homology regions. The left homology region corresponded to the 5′ UTR of the HBA1, and the right homology region corresponded to the 3′ UTR (FIG. 2A), starting immediately downstream of the sgRNA target site. sgRNA5-Cas9 RNPs were introduced by electroporation into thawed CD34+ HSPCs, immediately followed by addition of AAV6 carrying the PAH donor vector. In addition, we developed a co-expression cassette containing the codon-optimized PAH transgene, a T2A sequence, and a YFP marker gene driven by the constitutive promoter SFFV (FIG. 2A), with the sequence listed as SEQ ID NO: 12. Upon expression the T2A peptide allows separate translation of PAH and YFP. Cells were plated in HSPC media for 2 days, and then RBC differentiation was initiated by transferring the cells into phase 1 RBC media (FIG. 2B). The integration of the transgene PAH reached 20.95% and 22.47% of targeted alleles at day 0 and day 14, respectively in the presence of sgRNA, Cas9 protein, and donor template (FIG. 2C). Integration of the transgene SFFV-PAH-YFP (bulk) was 19.21% of targeted alleles, while YFP (sorted) reached 60.72% (FIG. 2C). Integration of the transgene did not affect differentiation of the cells, with close to 100% of all the tested sample cells showing RBC differentiation (FIG. 2D).

At day 14 of RBC differentiation, cells were also harvested for qualitative analysis of protein expression by western blot targeted with either HBA1-PAH or SFFV-PAH-T2A-YFP. Compared to the GAPDH reference protein, high levels of PAH expression in both the HBA1-PAH and SFFV-PAH-T2A-YFP vectors are observed in the bulk population of targeted cells post-differentiation (FIG. 2E). To validate the efficiency of T2A, the relative quantity of PAH-YFP fusion protein (˜78 kD) vs. PAH (51 kD) was evaluated by measuring the intensity of the two bands in the upper portion of the “PAH-T2A-YFP” column in the Western blot image (FIG. 2E). Roughly 98.9% of detected PAH protein was present in the correct “cleaved” form, suggesting that the T2A peptide sequence is highly efficient and does not meaningfully interfere with PAH expression.

We next examined in vitro PAH enzymatic activity. The assay is mostly based on the methods described in Martinez et a.l, Biochem J. 1995; and Yew et al., Mol Genet Metab 2013. Briefly, cells targeted with HBA1-PAH or SFFV-PAH-YFP were harvested at d14 of RBC differentiation (sorting for YFP+ cells were done at d7 as described above). Lysates were obtained by freeze/thaw and centrifuged to remove debris. Lysate was combined with L-Phe, 6-MPH4 cofactor, DTT, and Catalase in Tris-HCl and incubated for 3 hours at 25 C. Trichloroacetic acid, 1-Nitroso-2-naphthol and Nitric Acid were added followed by 30 min incubation at 55 C. Tyrosine was quantified calorimetrically at 450 nm and the umol of Tyrosine calculated from a standard curve. HEPG2 cells (a hepatocellular carcinoma cell line, which endogenously express PAH) were assayed as a positive control. As shown in FIG. 2F, HBA1-PAH (34.02 umol Tyr/min/10⁹ cells) has significantly more PAH activity than SFFV-PAH-YFP (p<0.001 for both Bulk (2.055 umol Tyr/min/10⁹ cells) and Sorted (12.66 umol Tyr/min/10⁹ cells), suggesting that the HBA1 promoter is a robust driver of protein expression in erythroid cells, even for non-erythroid proteins such as PAH. HEPG2 cells exhibit approx. 1.29-fold more PAH activity (43.89 umol Tyr/min/10⁹ cells) than HBA1-PAH-targeted erythroid cells (FIG. 2F). HSCs were targeted with SFFV-PAH-YFP and differentiated into RBCs as previously described. On d7, cells were sorted for YFP, and both Bulk (unsorted) and Sorted cells were returned to culture. At d14 of RBC differentiation, cells were harvested and analyzed by FACS for % YFP+ of live cells (aka, % edited cells). As shown in FIG. 2G, Bulk (unsorted) cells exhibit up to 25.5% YFP+ of live cells, while sorted cells can reach up to 85.03%. Connecting lines indicate unique replicates (donors). The sample denoted with an asterisk (*) corresponds to data shown in FIG. 2F.

### Example 2. Transplantation of Genetically Edited Townes Mouse HSPCs into PAH-Deficient Mice

CD34+ HSPC are isolated from the bone marrow of Townes mice and expanded ex vivo. The cells are then edited by introducing RNPs containing sg5 sgRNA and Cas9 by electroporation, and a donor template comprising left and right HBA1 homology regions surrounding a codon-optimized PAH transgene introduced using an AAV6 vector. The edited HSCs are allowed to recover for 1 week and then transplanted into irradiated homozygous PAH^{enu2/enu2} mice (FIG. 3).

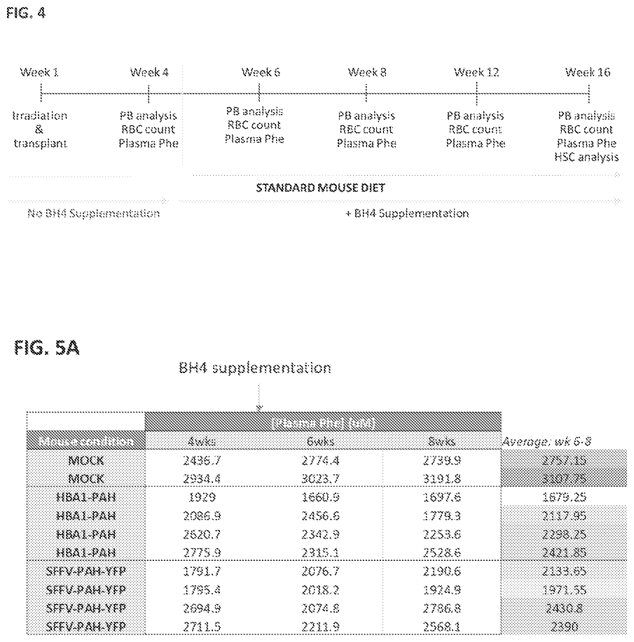

Following transplantation of the edited HSPCs, the mice are fed a standard mouse diet, with no BH4 supplementation. At four weeks they are started on daily oral BH4 supplementation in their drinking water. Plasma phenylalanine, peripheral blood analysis, and complete blood count is measured before and every 4 weeks after starting BH4 supplementation (FIG. 4).

Homozygous PAH^{enu2/enu2} mice were irradiated at 8 wks old with 8Gray Total Body Irradiation (TBI). Townes mouse HSCs either unedited (Mock), edited with HBA1-PAH (HBA1-PAH), or edited with SFFV-PAH-YFP (SFFV-PAH-YFP) were transplanted via Retro-orbital injection the same day. Blood was drawn via retro-orbital bleeds at week 4, 6, and 8. Plasma was separated from whole blood by centrifugation and the plasma [Phe] (uM) was determined by LC/MS. Data for individual mice across weeks 4-8 are shown in FIG. 5A. BH4 cofactor supplementation in drinking water (20 mg/kg/day dosing) was initiated 1-day after the wk4 samples were obtained. The last column in FIG. 5A shows the average [Phe]ₚₗₐₛₘₐ for wks 6 and 8 for individual mice. As also shown in FIG. 5B, “Mock” unedited HSCs exhibited an average of 2932 uM [Phe]ₚₗₐₛₘₐ, HSCs edited with HBA1-PAH and HSCs edited with SFFV-PAH-YFP (bulk) had an average of 2232 uM (p=0.02 vs. Mock) and 2129 uM (p=0.04 vs. Mock) [Phe]ₚₗₐₛₘₐ, respectively. The in vivo PAH data indicates transplant of edited mHSCs expressing PAH driven either by HBA1 (RBC-specific) or SFFV (in all HSC lineages) leads to a significant reduction in [Phe]_{Plasma} in mice with a severe PKU phenotype.

### Example 3. Development and Introduction of Improved Factor IX Transgenes

In this example, we used the Cas9/AAV6 genome editing method to mediate site-specific integration of various codon-optimized Factor IX (FIX) minigenes with improved properties into the HBA1 locus while leaving the virtually identical HBA2 gene unperturbed. We found that this process allowed us to replace the entire coding region of HBA1 with the FXI transgene at high frequencies, which allowed high levels of FIX expression in differentiated RBCs.

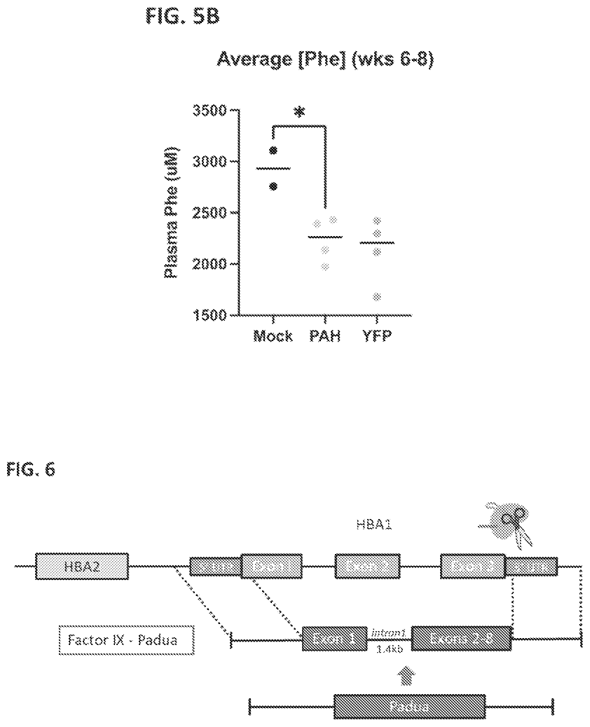

We generated a Factor IX transgene with a “Padua” mutation (R338L) and integrated it into a donor template, with flanking right and left HBA1 homology regions (SEQ ID NO:3 and SEQ ID NO:4). The left homology region corresponded to the 5′ UTR of the HBA1, and the right homology region corresponded to the 3′ UTR, starting immediately downstream of the sgRNA target site (FIG. 6).

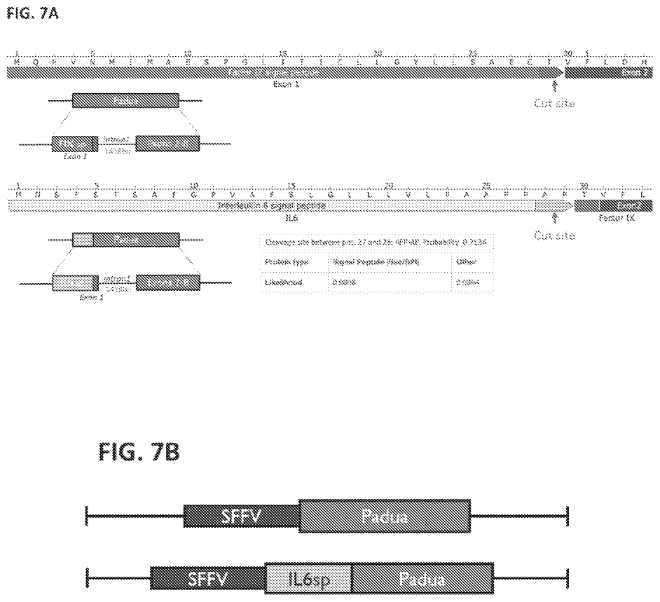

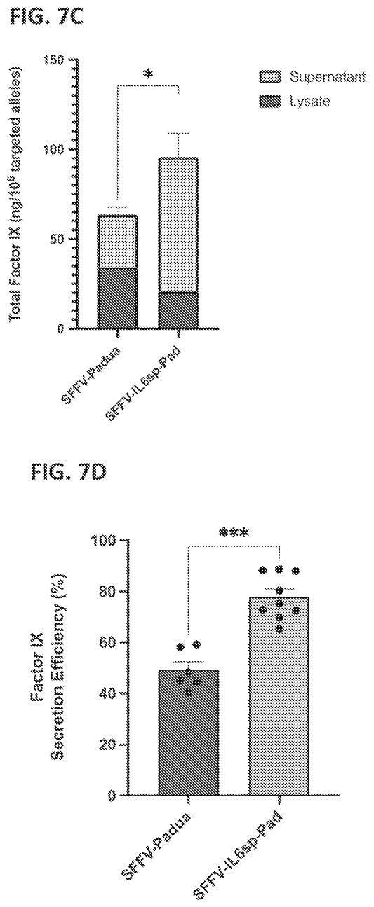

The efficiency of secretion was enhanced by generating new secretion vectors comprising, e.g., a FIX transgene comprising an IL-6 signal peptide (FIG. 7A), operably linked to a silencing-prone spleen focus forming virus (SFFV) promoter (FIG. 7B). The Padua vector described in FIG. 6 was modified by addition of the constitutive SFFV promoter immediately upstream of the start codon for Padua, resulting the new constitutive expression vector SFFV-Padua (FIG. 7B). The transgene is flanked by a 400 bp LHA (66 bp of the original L homology arm was removed, comprising the HBA1 5′UTR and a 400 bp RHA (unchanged). The other constitutive expression vector for HSC targeting experiment SFFV-IL6-Padua was constructed by inserting the SFFV promoter immediately upstream of the 87 bp 1L6-signal peptide (FIG. 7B).

To compare the two signal peptides, CD34⁺ HSPCs were targeted as described herein with Cas9/sgRNA and an AAV6 carrying either SFFV-Padua or SFFV-116-Padua. Addition of the SFFV promoter allows all of the optimization experiments to be performed over short term (7 day) HSC cultures, rather than over the course of a 2-week RBC differentiation. After targeting, cells were then cultured in HSC media for 7 days with a media change at day 3 and day 6. On day 7, 24 h after the day 6 media change, samples were centrifuged and the cell culture media (supernatant) and whole cell pellet were harvested for analysis. The amount of factor IX protein contained in the culture supernatants and cell lysates was quantified with a factor IX ELISA (FIGS. 7C-7D). The protein concentrations in the supernatant and lysate were normalized by % targeted alleles, to account for variability in targeting efficiency across different donor HSCs (biological replicates). Bar sections depict the proportion of secreted vs. cytoplasmic Factor IX. This normalization allows for direct (intra-sample) comparison of protein concentration in the secreted vs cellular fractions (FIG. 7C). Secretion efficiency was calculated by dividing the quantity of Factor IX in the secreted fraction by the total (secreted and cellular) quantity. The IL-6 signal peptide significantly enhanced secretion by 1.58-fold over the native Factor IX signal peptide (mean secretion efficiency 77.93% and 49.30%, respectively; p<0.001, unpaired one-tailed T-test) (FIG. 7D).

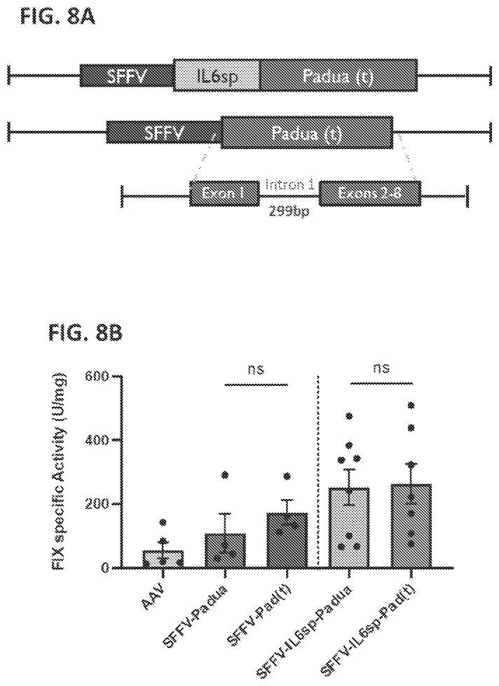

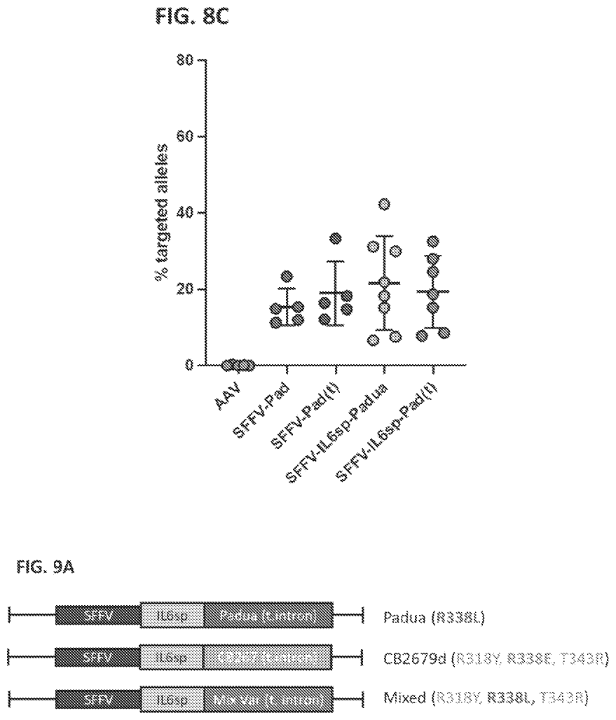

The FIX transgenes used comprise exon 1 of the FIX gene, followed by a portion of intron 1 and exons 2-8 (FIG. 8A). The full-length intron is 6.2 kb, much greater than the packaging capacity of AAV6. A 1.4 kb portion of intron 1 retains the positive regulatory effects of intron 1, but further truncation of the intron to 299 bp was tested with the FIX transgenes to reduce the overall length of the transgene, to increase the efficiency of, e.g., gene transfer, expression, and/or integration and/or to allow additional elements (e.g., selection markers) to be added to the vector without impairing efficiency. (FIG. 8C). Factor IX specific activity was measured on day 7 of HSC culture (24 h in fresh media). Factor IX concentration in the media was quantified by ELISA. Protein activity was measured with a modified aPTT assay, mixing conditioned culture media with factor IX deficient plasma and then measuring clotting activity on a coagulation analyzer. Specific activity was calculated as Units of factor IX activity per mg protein. All cells receiving FIX transgenes (SFFV-Padua, SFFV-Pad(t), SFFV-IL6-Padua, and SFFV-IL6-Pad(t)) showed high FIX specific activities, while no significant differences in specific activity were observed between truncated vs. original-length intron constructs (SFFV-Padua vs. SFFV-Pad(t), or SFFV-IL6-Padua vs. SFFV-IL6-Pad(t)) (FIG. 8B). Allelic targeting analysis by ddPCR showed no significant differences in targeting rates between any conditions (paired T-tests, two tailed, p>0.05), indicating targeting rates were unaffected by intron size (FIG. 8C).

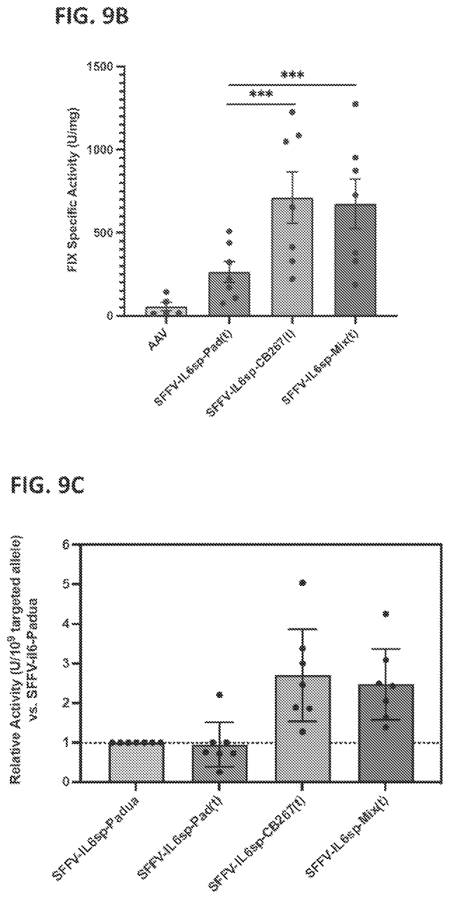

Nair et al. (Blood (2021) 137 (21): 2902-2906)) identified a FIX mutant, CB2679d, which has three mutations relative to wild-type FIX (R318Y, R338E, T343R) and 2-3 times higher activity relative to the Padua mutant. A novel variant was generated, comprising mutations R318Y, R338L, and T343R from the CB2679d and the Padua variants. Different FIX variants were introduced into hematopoietic stem cells (HSCs) using secretion vectors comprising an SFFV promoter and an IL-6 signal peptide (FIG. 9A). Both the CB2679d mutant (SFFV-IL6-CB267(t)) and the FIX Mixed variant (SFFV-IL6-Mix(t)) displayed significantly greater FIX specific activity than the Padua mutant (SFFV-IL6-pad(t)) (FIG. 9B). The CB2679d mutant and the FIX Mixed variant led to a 2.698- and 2.474-fold increase, respectively, in activity over the Padua mutant after normalizing by allelic targeting rates (FIG. 9C). There is no significant difference in FIX activity between the CB2679d mutant and the FIX Mixed variant (FIG. 9C). The integrations of the transgenes SFFV-IL6-CB267(t) and SFFV-IL6-Mix(t) in HSCs reached 29.47% and 28.7% targeted alleles respectively, which were significantly higher than the integration of the transgene SFFV-IL6-Pad(t) (19.38% targeted alleles) (FIG. 9D).

### Example 4. FIX-Variant Experiments in RBCs

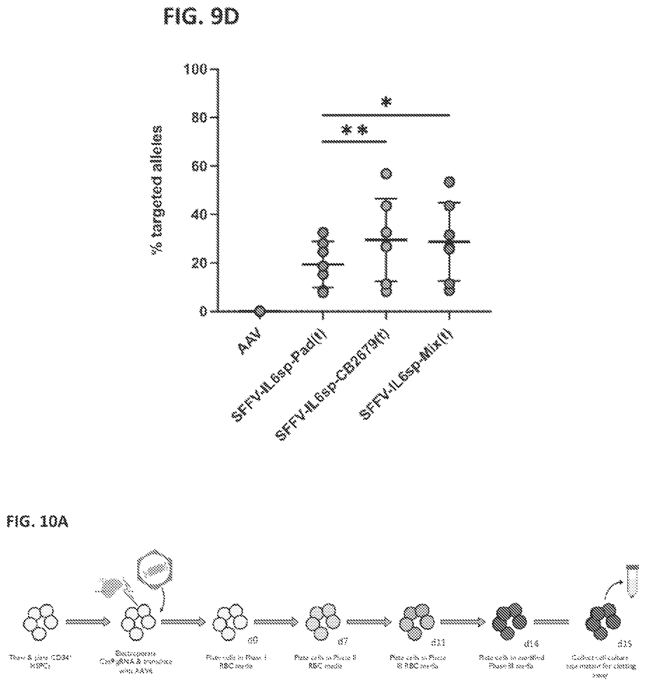

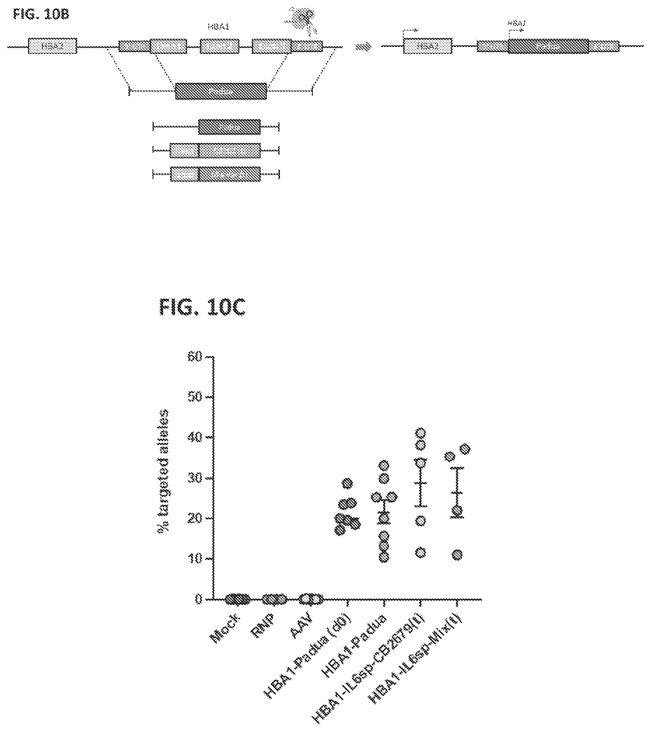

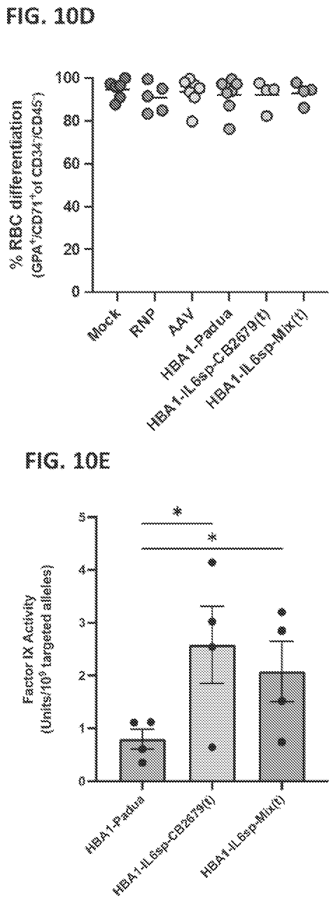

After the Factor IX vectors with HSC experiments were optimized, next we tested FIX virants in RBCs. Briefly, human CD34+ HSPCs were targeted with a combined AAV6/Cas9 strategy, cultured for 2 days in HSC media, and then differentiated down the erythroid lineage with a 3-phase, 14-day differentiation protocol. As described in FIG. 10A, on day 14 cells were counted, spun down, washed with PBS and then replated at ˜3⁶ cells/mL in modified Phase 3 RBC media (lacking human serum and plasma). 24 hours later the cell culture supernatant was collected to measure Factor IX activity using a modified aPTT assay.

Three constructs were designed for RBC-specific expression of FIX variants: Padua (control vector), IL6-CB2679d (truncated intron), and IL6-Mixed Variant (truncated intron), all driven by the endogenous HBA1 promoter (FIG. 10B). The 3 constructs are essentially RBC-specific versions of the constructs shown in FIG. 9A, as they notably lack SFFV and are thus turned on in response to HBA1.

To determine whether RBCs are capable of expressing Factor IX (Padua variant) at high levels in vitro, a purified population of human CD34+ HSPCs was targeted with an AAV6 vector carrying a hyperactive factor IX transgene for targeted replacement of HBA1. Cells were differentiated in vitro and analyzed for targeting rates by ddPCR, and RBC differentiation was monitored by flow cytometry. Allelic targeting analysis by ddPCR performed as the method described in FIG. 2C on samples taken at day 14 of the RBC differentiation. There is no significant difference between the groups HBA1-Padua (21.6%), HBA1-IL6-CB2679(t) (27.56%), and HBA1-IL6-Mix(t) (26.38%) respectively (p>0.05 for all comparisons) (FIG. 10C). RBC differentiation analysis by FACS on day 14, as the method described in FIG. 2D. There is no significant difference between the groups HBA1-Padua, HBA1-IL6-CB2679(t), and HBA1-IL6-Mix(t) (FIG. 10D). To measure FIX activity from the RBC samples in vitro, on d14 cells were counted, pelleted, washed with PBS 1×, and replated at ˜1 to 3e6 cells/mL in modified Phase 3 RBC media (lacking human serum and plasma, to eliminate background coagulation factor activity). After 24 hours, cell culture supernatant was collected to measure Factor IX activity by a modified aPTT assay. Mean FIX Activity (indicated as U/10⁹ targeted alleles) of RBC sampled from d15 cell culture supernatant were measured. Both the HBA1-IL6-CB2679(t) and HBA1-IL6-Mix groups showed significant increase in factor IX activity, leading to a 2.586- and 2.079-fold higher, respectively, over HBA1-Padua (FIG. 10E).

### Example 5. Exemplary Hemophilia B Mouse Experiments

The B6.129P2-F9^{tm1Dws}/J mouse model (factor IX knockout) can be used to evaluate FIX virants as illustrated in FIG. 11. Townes HSCs are harvested, expanded, edited and transplanted as previously described for the PKU mouse experiments. The radiation dose can be titrated to assess if disease correction can be achieved with half or low dose (or potentially no) radiation.

### Example 6. tEPOR Enrichment of HBA1-Factor IX Targeted RBCs

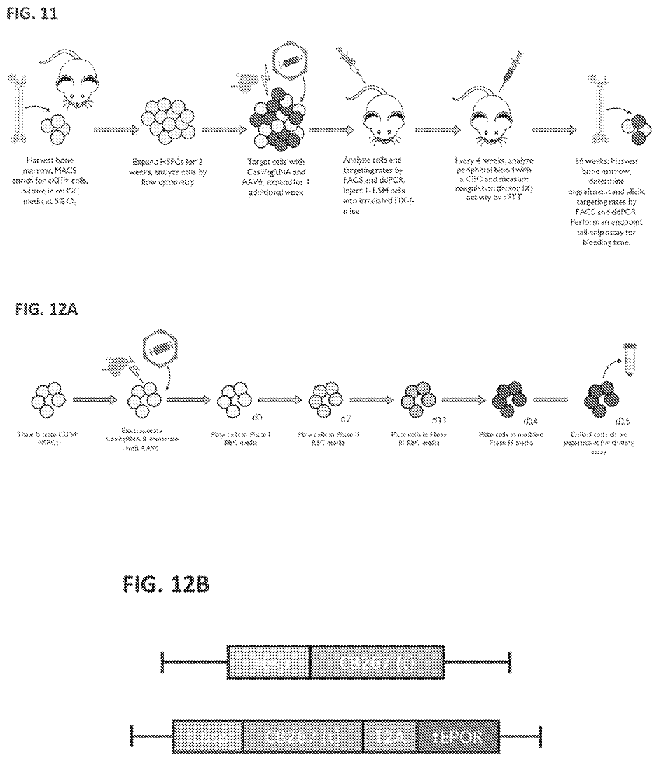

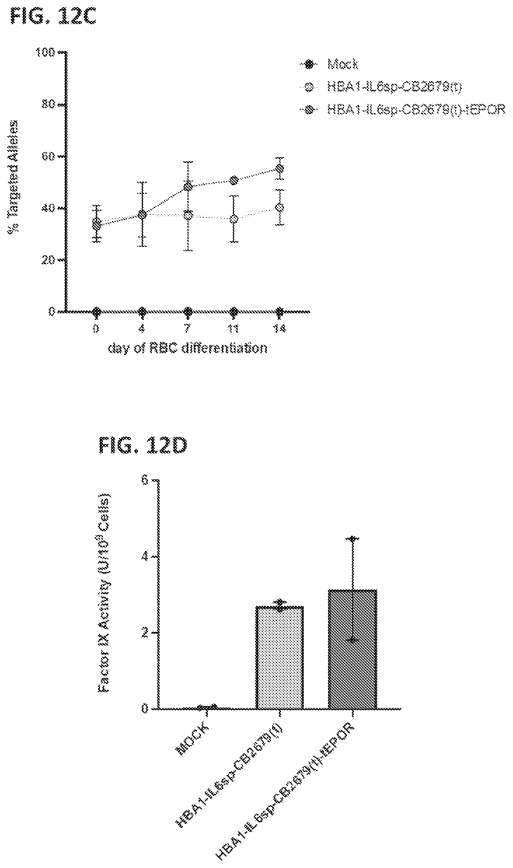

As illustrated in FIG. 12A, human CD34+ HSPCs were targeted with a combined AAV6/Cas9 strategy, cultured for 2 days in HSC media, and then differentiated down the erythroid lineage with a 3-phase, 14-day differentiation protocol. On day 14 cells were counted, spun down, washed with PBS and then replated at ˜3″ cells/mL in modified Phase 3 RBC media (lacking human serum and plasma). 24 hours later the cell culture supernatant was collected to measure Factor IX activity using a modified aPTT assay.

Two vectors were constructed and tested in this study (FIG. 12B). The first vector HBA1-IL6-CB2679(t) was the same vector from the earlier FIX Variant RBC experiments in Example 4. The second vector HBA1-1L6-CB2679(t)-T2A-tEPOR is a modification of the first that includes a T2A sequence immediately downstream of the CB2679 FIX variant gene (but prior to the stop codon), followed by a truncated erythropoietin receptor gene sequence. This truncated EPO receptor is more sensitive than the wild-type receptor to EPOR signaling and can provide a selective advantage to targeted HSCs undergoing RBC differentiation such that cells targeted with this vector are selectively enriched within the differentiating population. With this construct both FIX and tEPOR expression are driven by the endogenous HBA1 promoter, restricting expression to committed RBC progenitors.

Allelic targeting analysis and tEPOR enrichment. Cells targeted with these vectors were differentiated. A sample of cells were taken from culture at days 0, 4, 7, 11, and 14 of the differentiation for ddPCR analysis of allelic targeting at the HBA1 locus (FIG. 12C). IL6-CB2679-tEPOR targeted alleles enriched 1.7-fold from day 0 (33.1%) to day 14 (55.2%), and 1L6-CB2679-tEPOR had 1.4× higher targeting than IL6-CB2679 on d14 (55.2% vs. 40.3%), indicating the truncated EPO receptor can enrich targeted HSCs undergoing RBC differentiation. Factor IX Activity (U/10⁹ cells/mL) determined using a modified aPTT coagulation assay with day 15 cell culture media (FIG. 12D). IL6-CB2679-tEPOR had 1.2-fold greater Factor IX activity than IL6-CB2679 (3.139 vs. 2.716), further confirming the enrichment effects of the truncated EPO receptor.

### Example 7. Discussion

The targeted integration of our transgenes offers multiple advantages over previous methods. For example, it offers an improvement over lentiviral gene therapies which carry the risk of insertional mutagenesis. Further, by driving production of therapeutic proteins from the HBA1 locus, we can also guarantee robust expression restricted solely to the erythroid lineage, sparing HSC function and mitigating the risk of off-target or off-tissue effects. With our ex vivo editing strategy, we also avoid the major limitation of most current AAV gene therapy candidates, which commonly rely on systemic administration of AAV for in vivo transduction of hepatocytes: up to 60% of adults have pre-existing immunity against AAV, making them ineligible to receive this type of treatment. Additionally, systemic administration of AAV elicits a robust immune response that precludes re-dosing for patients should the efficacy of these gene therapies wane over time. Lastly, due to our transgene optimizations as described above for our hemophilia B therapy, we have increased the secretion and activity of factor IX several-fold compared to Padua, the factor IX cDNA transgene currently being used in hemophilia B gene therapy clinical trials. Thus, we believe we can achieve therapeutic levels of factor IX activity with even low levels of HSC engraftment post-transplant, which may be attainable with minimally toxic or antibody-mediated bone marrow conditioning. This would improve the safety of our approach over conventional ex vivo HSC editing/transplant strategies.

### Example 8. Methods

### AAV6 Vector Design, Production, and Purification

All AAV6 vectors were cloned into the pAAV-MCS plasmid (Agilent Technologies, Santa Clara, CA, USA), which contains inverted terminal repeats (ITRs) derived from AAV2. Gibson Assembly Mastermix (New England Biolabs, Ipswich, MA, USA) was used for the creation of each vector as per manufacturer's instructions. All vectors have a left homology arm (LHA) flanking the 5′ UTR of HBA1 gene while the right homology arm (RHA) immediately flanks downstream of the cut site. The LHA of all of the HBA1 vectors are 466 bp except those that include SFFV which have a 400 bp LHA, and the RHA of every vector is 400 bp.

293T cells (Life Technologies, Carlsbad, CA, USA) were seeded in ten 15 cm² dishes with 13-15×10⁶ cells per plate. 24 h later, each dish was transfected with a standard polyethylenimine (PEI) transfection of 6 μg ITR-containing plasmid and 22 μg pDGM6, which contains the AAV6 cap genes, AAV2 rep genes, and Ad5 helper genes. After a 48-72 h incubation, cells were lysed by 3 freeze-thaw cycles, treated with benzonase (Thermo Fisher Scientific, Waltham, MA, USA) at 250 U/mL, and the vector was then purified through an iodixanol gradient centrifugation at 48,000 RPM for 2.25 h at 18° C. Afterwards, full capsids were isolated at the 40-58% iodixanol interface and then stored at 80° C. until further use. As an alternative method, AAVPro Purification Kit (All Serotypes) (Takara Bio USA, Mountain View, CA, USA) were also used following the 48-72 h incubation period, to extract full AAV6 capsids as per manufacturer's instructions. AAV6 vectors were titered using ddPCR to measure number of vector genomes as previously described.

### Culturing of CD34⁺ HSPCs

Human CD34+ HSPCs were sourced from fresh cord blood, frozen cord blood and Plerixafor- and/or G-CSF-mobilized peripheral blood (AllCells, Alameda, CA, USA and STEMCELL Technologies, Vancouver, Canada). CD34⁺ HSPCs were cultured at 1×10⁵-5×10⁵ cells/mL in CellGenix GMP SCGM (CellGenix, Freiburg, Germany) base medium supplemented with stem cell factor (SCF) (100 ng/mL), thrombopoietin (TPO) (100 ng/mL), FLT3-ligand (100 ng/mL), IL-6 (100 ng/mL), UM171 (35 nM), 20 mg/mL streptomycin, and 20 U/mL penicillin. The cell incubator conditions were 37° C., 5% CO₂, and 5% O₂.

### Genome Editing of CD34⁺ HSPCs

Chemically modified sgRNAs used to edit CD34⁺ HSPCs at HBA1 were purchased from Synthego (Menlo Park, CA, USA) and TriLink BioTechnologies (San Diego, CA, USA) and were purified by high-performance liquid chromatography (HPLC). The sgRNA modifications added were the 2′-O-methyl-3′-phosphorothioate at the three terminal nucleotides of the 5′ and 3′ ends described previously. The target sequence for the sgRNA was as follows: sg5: 5′-GGCAAGAAGCATGGCCACCG-3′ (SEQ ID NO:1). All Cas9 protein (Alt-R S.p. HiFi Cas9 Nuclease V3) used was purchased from Integrated DNA Technologies (Coralville, Iowa, USA). The RNPs were complexed at a Cas9:sgRNA molar ratio of 1:2.5 at 25° C. for 10 min prior to electroporation. CD34⁺ cells were resuspended in P3 buffer (Lonza, Basel, Switzerland) with complexed RNPs and electroporated using the Lonza 4D Nucleofector (program DZ-100). Cells were plated at 1×10⁵ cells/mL following electroporation in the cytokine-supplemented media described previously. Immediately following electroporation, AAV6 was supplied to the cells at 5×10³ vector genomes/cell based on titers determined by ddPCR

### Gene Targeting Analysis by Flow Cytometry

4-8 d post-targeting with fluorescent integration vectors, CD34⁺ HSPCs were harvested and the percentage of edited cells was determined by flow cytometry. Cells were analyzed for viability using Ghost Dye Red 780 (Tonbo Biosciences, San Diego, CA, USA) and reporter expression was assessed using either the Accuri C6 flow cytometer (BD Biosciences, San Jose, CA, USA) or FACS Aria II (BD Biosciences, San Jose, CA, USA). The data was subsequently analyzed using FlowJo (FlowJo LLC, Ashland, OR, USA).

In Vitro Differentiation of CD34⁺ HSPCs into Erythrocytes

Following targeting, HSPCs were cultured for 14-16 d at 37° C. in 5% O₂ and 5% CO₂ in SFEM II medium (STEMCELL Technologies, Vancouver, Canada) as previously described (35, 36). SFEMII base medium was supplemented with 100 U/mL penicillin-streptomycin, 10 ng/mL SCF, 1 ng/mL IL-3 (PeproTech, Rocky Hill, NJ, USA), 3 U/mL erythropoietin (eBiosciences, San Diego, CA, USA), 200 μg/mL transferrin (Sigma-Aldrich, St. Louis, MO, USA), 3% antibody serum (heat-inactivated from Atlanta Biologicals, Flowery Branch, GA, USA), 2% human plasma (umbilical cord blood), 10 μg/mL insulin (Sigma-Aldrich, St. Louis, MO, USA) and 3 U/mL heparin (Sigma-Aldrich, St. Louis, MO, USA). In the first phase, d 0-7 (day zero being 2d post-targeting) of differentiation, cells were cultured at 1×10⁵ cells/mL. In the second phase, d7-10, cells were maintained at 1-10 cells/mL, and IL-3 was removed from the culture. In the third phase, d11-16, cells were cultured at 1×10⁶ cells/mL, and transferrin was increased to 1 mg/mL within the culture medium.

### Immunophenotyping of Differentiated Erythrocytes

HSPCs subjected to the above erythrocyte differentiation were analyzed at d14-16 for erythrocyte lineage-specific markers using a FACS Aria II (BD Biosciences, San Jose, CA, USA). Edited and non-edited cells were analyzed by flow cytometry using the following antibodies: hCD45 V450 (HI30; BD Biosciences, San Jose, CA, USA), hCD34 APC (561; BioLegend, San Diego, CA, USA), hCD71 PE-Cy7 (OKT9; Affymetrix, Santa Clara, CA, USA), and hCD235a PE (GPA) (GA-R2; BD Biosciences, San Jose, CA, USA).

CD34⁺ HSPC Transplantation into Immunodeficient Mice

Eight to ten-week-old female PAH^{enu2/enu2} mice (Jackson Laboratory, Bar Harbor, ME, USA) are irradiated using 9.5 Gy of radiation immediately prior to transplantation with targeted HSPCs (2d post-targeting) via retroorbital injections. Approximately 2.5-10⁵-1.5×10⁶ targeted HSPCs are injected using an insulin syringe with a 27G, 0.5 inch (12.7 mm) needle. Sample sizes used in this study are within the range reported in previous Cas9/AAV6-mediated genome editing studies (21-23).

### Statistical Analysis

All data points presented in the figures were taken from distinct treatment groups rather than repeated measurements of the same treatment. Sample sizes used in this study were within the range reported in previous Cas9/AAV6-mediated genome editing studies. No data exclusion criteria were established prior to the execution of any experiments reported in this paper, and no data were excluded following conclusion of the experiments. Where possible, all experiments were replicated across a minimum of three or more CD34⁺ HSPC donors. All statistical tests on experimental groups were done using Prism7 GraphPad Software. Two-tailed unpaired t tests were used to determine statistical differences among treatment groups. Sample variance was determined for all treatment groups, and where found to be unequal, Welch's t test also confirmed statistical significance.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### 7. Exemplary Embodiments

Exemplary embodiments provided in accordance with the presently disclosed subject matter include, but are not limited to, the claims and the following embodiments:

Embodiment 1. A method of genetically modifying a hematopoietic stem and progenitor cell (HSPC) from a subject, the method comprising:
introducing into the HSPC a guide RNA targeting the HBA1 or HBA2 locus, an RNA-guided nuclease, and a homologous donor template comprising a transgene encoding Factor IX, wherein
the RNA-guided nuclease cleaves the HBA1 or HBA2 locus, but not both, in the cell; the transgene is integrated into the genome by homology directed recombination (HDR) at the site of the cleaved HBA1 or HBA2 locus; and the integrated transgene directs the expression of Factor IX in the HSPC; and wherein
the Factor IX comprises an exogenous signal peptide;
the Factor IX comprises two or more amino acid substitutions relative to the wild type sequence shown as SEQ ID NO:13, wherein the two or more amino acid substitutions are selected from the group consisting of R318Y, R338E, R338L, and T343R; and/or
the transgene comprises a truncated intron 1 of the FIX gene.

Embodiment 2. The method of embodiment 1, wherein the method further comprises isolating the HSPC from the subject prior to the introducing of the guide RNA, RNA-guided nuclease, and homologous donor template.

Embodiment 3. The method of embodiment 1 or 2, wherein the target sequence of the guide RNA comprises the sg5 target sequence (SEQ ID NO:1), and wherein the RNA-guided nuclease cleaves the HBA1 locus.

Embodiment 4. The method of any one of embodiments 1 to 3, wherein the homologous donor template comprises an HBA1 left homology arm comprising the sequence of SEQ ID NO:3 or a subsequence thereof, and/or HBA1 right homology arm comprising the sequence of SEQ ID NO:4 or a subsequence thereof.

Embodiment 5. The method of embodiment 1 or 2, wherein the target sequence of the guide RNA comprises the sg2 target sequence (SEQ ID NO:2), and wherein the RNA-guided nuclease cleaves the HBA2 locus.

Embodiment 6. The method of any one of embodiments 1 to 5, wherein the subject has hemophilia B, and wherein the genetically modified HSPC expressing Factor IX is reintroduced into the subject.

Embodiment 7. The method of embodiment 6, wherein the reintroduction of the genetically modified HSPC into the subject improves one or more symptoms of the hemophilia B.

Embodiment 8. The method of any one of embodiments 1 to 7, wherein the expression of the integrated transgene is driven by an endogenous HBA1 or HBA2 promoter.

Embodiment 9. The method of any one of embodiments 1 to 7, wherein the expression of the integrated transgene is driven by an exogenous promoter.

Embodiment 10. The method of embodiment 9, wherein the exogenous promoter is the SFFV promoter.

Embodiment 11. The method of any one of embodiments 1 to 10, wherein the integrated transgene replaces the HBA1 or HBA2 coding sequence in the genome.

Embodiment 12. The method of any one of embodiments 1 to 11, wherein the exogenous signal peptide is an IL6 signal peptide.

Embodiment 13. The method of any one of embodiments 1 to 12, wherein the transgene further encodes a truncated EPO receptor (tEPOR) downstream in fusion with the Factor IX.

Embodiment 14. The method of embodiment 13, wherein the tEPOR is linked to the Factor IX through a T2A peptide sequence.

Embodiment 15. The method of any one of embodiments 1 to 14, wherein the amino acid substitutions comprise R318Y, R338L, and T343R.

Embodiment 16. The method of any one of embodiments 1 to 15, wherein the intron 1 is truncated by at least about 0.5, 1.0, 1.5, 2.0, 2.5, 3.0, 3.5, 4.0, 4.5, 4.6, 4.7, 4.8, 4.9, 5.0, 5.5, 5.6, 5.7, 5.8, 5.9, 6.0, or more kb relative to the full-length FIX intron 1.

Embodiment 17. The method of embodiment 16, wherein the intron is truncated by about 4.8 kb or about 5.9 kb.

Embodiment 18. The method of any one of embodiments 1 to 17, wherein the transgene is codon optimized.

Embodiment 19. The method of any one of embodiments 1 to 18, wherein the transgene comprises a sequence shown as SEQ ID NOS: 6-11 or a subsequence thereof, or a nucleotide sequence comprising at least about 70%, 75%, 80%, 85%, 90/o, 95%, 96%, 97%, 98%, 99%, or more identity to any of SEQ ID NOS: 6-11 or a subsequence thereof, wherein the Factor IX encoded by the transgene comprises two or more amino acid substitutions selected from the group consisting of R318Y, R338E, R338L, and T343R.

Embodiment 20. The method of embodiment 19, wherein the Factor IX encoded by the transgene comprises the amino acid substitutions R318Y, R338L, and T343R.

Embodiment 21. The method of any one of embodiments 1 to 20, wherein the guide RNA comprises one or more 2′-O-methyl-3′-phosphorothioate (MS) modifications.

Embodiment 22. The method of embodiment 21, wherein the 2′-O-methyl-3′-phosphorothioate (MS) modifications are present at the three terminal nucleotides of the 5′ and 3′ ends.

Embodiment 23. The method of any one of embodiments 1 to 22, wherein the RNA-guided nuclease is Cas9.

Embodiment 24. The method of embodiment 23, wherein the Cas9 is a high fidelity Cas9.

Embodiment 25. The method of any one of embodiments 1 to 24, wherein the guide RNA and the RNA-guided nuclease are introduced into the HSPC as a ribonucleoprotein (RNP) by electroporation.

Embodiment 26. The method of any one of embodiments 1 to 25, wherein the homologous donor template is introduced into the cells using a recombinant adeno-associated virus (rAAV) serotype 6 vector.

Embodiment 27. The method of any one of embodiments 1 to 26, further comprising a step in which the genetically modified HSPC is induced to differentiate in vitro into a red blood cell (RBC).

Embodiment 28. The method of any one of embodiments 1 to 27, wherein the subject is a human.

Embodiment 29. A FIX transgene comprising a sequence shown as any of SEQ ID NOS: 6-11 or a subsequence thereof, or a nucleotide sequence comprising at least about 70%, 75%, 80%, 85%, 90%, 95%, 96%, 97%, 98%, 99%, or more identity to any of SEQ ID NOS: 6-11 or a subsequence thereof, wherein the Factor IX encoded by the transgene comprises two or more amino acid substitutions selected from the group consisting of R318Y, R338E, R338L, and T343R relative to SEQ ID NO:13.

Embodiment 30. The FIX transgene of embodiment 29, wherein the Factor IX comprises the amino acid substitutions R318Y, R338L, and T343R.

Embodiment 31. The FIX transgene of embodiment 29 or 30, wherein the transgene comprises an IL6 signal peptide.

Embodiment 32. The FIX transgene of any one of embodiments 29 to 31, wherein the transgene comprises a truncated intron 1 of the FIX gene.

Embodiment 33. An HSPC comprising the FIX transgene of any one of embodiments 29 to 32.

Embodiment 34. The HSPC of embodiment 33, wherein the FIX transgene is integrated into the HSPC genome at the HBA1 or HBA2 locus, but not both.

Embodiment 35. The HSPC of embodiment 34, wherein the HSPC was modified using the method of any one of embodiments 1 to 28.

Embodiment 36. A red blood cell produced by inducing the differentiation in vitro of the genetically modified HSPC of any one of embodiments 33 to 35 into a red blood cell.

Embodiment 37. A method of genetically modifying a hematopoietic stem and progenitor cell (HSPC) from a subject, the method comprising:
introducing into the HSPC a guide RNA targeting the HBA1 or HBA2 locus, an RNA-guided nuclease, and a homologous donor template comprising a PAH transgene, wherein
the RNA-guided nuclease cleaves the HBA1 or HBA2 locus, but not both, in the cell; the transgene is integrated into the genome by homology directed recombination (HDR) at the site of the cleaved HBA1 or HBA2 locus; and the integrated PAH transgene directs the expression of phenylalanine hydroxylase in the HSPC; and wherein
the PAH transgene comprises the sequence shown as SEQ ID NO:5.

Embodiment 38. The method of embodiment 37, wherein the subject has phenylketonuria, and wherein the genetically modified HSPC expressing phenylalanine hydroxylase is reintroduced into the subject.

Embodiment 39. The method of embodiment 38, wherein the reintroduction of the genetically modified HSPC into the subject improves one or more symptoms of the phenylketonuria.

Embodiment 40. The method of any one of embodiments 37 to 39, wherein the method further comprises administering B14 to the subject.

What is claimed is:

1. A method of genetically modifying a hematopoietic stem and progenitor cell (HSPC) from a subject, the method comprising:
introducing into the HSPC a guide RNA targeting the HBA1 or HBA2 locus, an RNA-guided nuclease, and a homologous donor template comprising a transgene encoding Factor IX, wherein
the RNA-guided nuclease cleaves the HBA1 or HBA2 locus, but not both, in the cell; the transgene is integrated into the genome by homology directed recombination (HDR) at the site of the cleaved HBA1 or HBA2 locus; and the integrated transgene directs the expression of Factor IX in the HSPC; and wherein
the Factor IX comprises an exogenous signal peptide;
the Factor IX comprises two or more amino acid substitutions relative to the wild type sequence shown as SEQ ID NO:13, wherein the two or more amino acid substitutions are selected from the group consisting of R318Y, R338E, R338L, and T343R; and/or
the transgene comprises a truncated intron 1 of the FIX gene.

2. The method of claim 1, wherein the method further comprises isolating the HSPC from the subject prior to the introducing of the guide RNA, RNA-guided nuclease, and homologous donor template.

3. The method of claim 1 or 2, wherein the target sequence of the guide RNA comprises the sg5 target sequence (SEQ ID NO:1), and wherein the RNA-guided nuclease cleaves the HBA1 locus.

4. The method of claim 1, wherein the homologous donor template comprises an HBA1 left homology arm comprising the sequence of SEQ ID NO:3 or a subsequence thereof, and/or HBA1 right homology arm comprising the sequence of SEQ ID NO:4 or a subsequence thereof.

5. The method of claim 1 or 2, wherein the target sequence of the guide RNA comprises the sg2 target sequence (SEQ ID NO:2), and wherein the RNA-guided nuclease cleaves the HBA2 locus.

6. The method of claim 1, wherein the subject has hemophilia B, and wherein the genetically modified HSPC expressing Factor IX is reintroduced into the subject.

7. The method of claim 6, wherein the reintroduction of the genetically modified HSPC into the subject improves one or more symptoms of the hemophilia B.

8. The method of claim 1, wherein the expression of the integrated transgene is driven by an endogenous HBA1 or HBA2 promoter.

9. The method of claim 1, wherein the expression of the integrated transgene is driven by an exogenous promoter.

10. The method of claim 9, wherein the exogenous promoter is the SFFV promoter.

11. The method of claim 1, wherein the integrated transgene replaces the HBA1 or HBA2 coding sequence in the genome.

12. The method of claim 1, wherein the exogenous signal peptide is an IL6 signal peptide.

13. The method of claim 1, wherein the transgene further encodes a truncated EPO receptor (tEPOR) downstream in fusion with the Factor IX.

14. The method of claim 13, wherein the tEPOR is linked to the Factor IX through a T2A peptide sequence.

15. The method of claim 1, wherein the amino acid substitutions comprise R318Y, R338L, and T343R.

16. The method of claim 1, wherein the intron 1 is truncated by at least about 0.5, 1.0, 1.5, 2.0, 2.5, 3.0, 3.5, 4.0, 4.5, 4.6, 4.7, 4.8, 4.9, 5.0, 5.5, 5.6, 5.7, 5.8, 5.9, 6.0, or more kb relative to the full-length FIX intron 1.

17. The method of claim 16, wherein the intron is truncated by about 4.8 kb or about 5.9 kb.

18. The method of claim 1, wherein the transgene is codon optimized.

19. The method of claim 1, wherein the transgene comprises a sequence shown as SEQ ID NOS: 6-11 or a subsequence thereof, or a nucleotide sequence comprising at least about 70%, 75%, 80%, 85%, 90%, 95%, 96%, 97%, 98%, 99%, or more identity to any of SEQ ID NOS: 6-11 or a subsequence thereof, wherein the Factor IX encoded by the transgene comprises two or more amino acid substitutions selected from the group consisting of R318Y, R338E, R338L, and T343R.

20. The method of claim 19, wherein the Factor IX encoded by the transgene comprises the amino acid substitutions R318Y, R338L, and T343R.

21. The method of claim 1, wherein the guide RNA comprises one or more 2′-O-methyl-3′-phosphorothioate (MS) modifications.

22. The method of claim 21, wherein the 2′-O-methyl-3′-phosphorothioate (MS) modifications are present at the three terminal nucleotides of the 5′ and 3′ ends.

23. The method of claim 1, wherein the RNA-guided nuclease is Cas9.

24. The method of claim 23, wherein the Cas9 is a high fidelity Cas9.

25. The method of claim 1, wherein the guide RNA and the RNA-guided nuclease are introduced into the HSPC as a ribonucleoprotein (RNP) by electroporation.

26. The method of claim 1, wherein the homologous donor template is introduced into the cells using a recombinant adeno-associated virus (rAAV) serotype 6 vector.

27. The method of claim 1, further comprising a step in which the genetically modified HSPC is induced to differentiate in vitro into a red blood cell (RBC).

28. The method of claim 1, wherein the subject is a human.

29. A FIX transgene comprising a sequence shown as any of SEQ ID NOS: 6-11 or a subsequence thereof, or a nucleotide sequence comprising at least about 70%, 75%, 80%, 85%, 90%, 95%, 96%, 97%, 98%, 99%, or more identity to any of SEQ ID NOS: 6-11 or a subsequence thereof, wherein the Factor IX encoded by the transgene comprises two or more amino acid substitutions selected from the group consisting of R318Y, R338E, R338L, and T343R relative to SEQ ID NO:13.

30. The FIX transgene of claim 29, wherein the Factor IX comprises the amino acid substitutions R318Y, R338L, and T343R.

31. The FIX transgene of claim 29 or 30, wherein the transgene comprises an IL6 signal peptide.

32. The FIX transgene of claim 29, wherein the transgene comprises a truncated intron 1 of the FIX gene.

33. An HSPC comprising the FIX transgene of claim 29.

34. The HSPC of claim 33, wherein the FIX transgene is integrated into the HSPC genome at the HBA1 or HBA2 locus, but not both.

35. The HSPC of claim 34, wherein the HSPC was modified using the method of any one of claims 1 to 28.

36. A red blood cell produced by inducing the differentiation in vitro of the genetically modified HSPC of claim 33 into a red blood cell.

37. A method of genetically modifying a hematopoietic stem and progenitor cell (HSPC) from a subject, the method comprising:
introducing into the HSPC a guide RNA targeting the HBA1 or HBA2 locus, an RNA-guided nuclease, and a homologous donor template comprising a PAH transgene, wherein
the RNA-guided nuclease cleaves the HBA1 or HBA2 locus, but not both, in the cell; the transgene is integrated into the genome by homology directed recombination (HDR) at the site of the cleaved HBA1 or HBA2 locus; and the integrated PAH transgene directs the expression of phenylalanine hydroxylase in the HSPC; and wherein
the PAH transgene comprises the sequence shown as SEQ ID NO:5.

38. The method of claim 37, wherein the subject has phenylketonuria, and wherein the genetically modified HSPC expressing phenylalanine hydroxylase is reintroduced into the subject.

39. The method of claim 38, wherein the reintroduction of the genetically modified HSPC into the subject improves one or more symptoms of the phenylketonuria.

40. The method of claim 37, wherein the method further comprises administering BH4 to the subject.


SEQUENCE LISTING (21 sequences)

SEQ ID NO: 1 - DNA, 20 bp, synthetic construct
  source 1..20: mol_type = other DNA
ggcaagaagcatggccaccg

SEQ ID NO: 2 - DNA, 20 bp, synthetic construct
  source 1..20: mol_type = other DNA
ggcaggaggaacggctaccg

SEQ ID NO: 3 - DNA, 466 bp, synthetic construct
  source 1..466: mol_type = other DNA
ccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccacc

SEQ ID NO: 4 - DNA, 400 bp, synthetic construct
  source 1..400: mol_type = other DNA
tggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactct

SEQ ID NO: 5 - DNA, 1359 bp, synthetic construct
  source 1..1359: mol_type = other DNA
atgagcaccgccgtgctggagaatcctggcctgggcagaaagctgagcgacttcggccaggagacatcctacatcgaggataactgcaatcagaacggcgccatctccctgatcttctctctgaaggaggaagtgggcgccctggcaaaggtgctgcgcctgtttgaggagaatgatgtgaacctgacccacatcgagtcccggccttctagactgaagaaggacgagtatgagttctttacccacctggataagcggtctctgccagccctgacaaacatcatcaagatcctgcgccacgatatcggagcaaccgtgcacgagctgagcagggacaagaagaaggataccgtgccctggttccctcggacaatccaggagctggacagatttgccaatcagatcctgtcctacggagcagagctggacgcagatcaccctggcttcaaggatccagtgtatagggcccggagaaagcagtttgccgacatcgcctacaactataggcacggacagccaatccctagggtggagtacatggaggaggagaagaagacctggggcacagtgttcaagaccctgaagtccctgtacaagacacacgcctgctacgagtataatcacatcttccccctgctggagaagtattgtggctttcacgaggacaacatccctcagctggaggacgtgagccagttcctgcagacctgtacaggctttaggctgaggccagtggcaggactgctgagctccagggatttcctgggaggactggccttcagagtgtttcactgcacccagtacatcaggcacggcagcaagccaatgtatacaccagagcccgatatctgtcacgagctgctgggccacgtgcccctgtttagcgaccggtccttcgcccagttttctcaggagatcggactggcaagcctgggagcacccgatgagtacatcgagaagctggccaccatctattggttcacagtggagtttggcctgtgcaagcagggcgactccatcaaggcatacggagcaggcctgctgtctagctttggcgagctgcagtattgtctgtctgagaagccaaagctgctgcccctggagctggagaagaccgccatccagaattacaccgtgacagagttccagcccctgtactatgtggccgagagctttaatgacgccaaggagaaggtgagaaacttcgccgccacaatccctaggccattttccgtgcgctacgacccttatacccagaggatcgaggtgctggataatacacagcagctgaagatcctggccgactctatcaacagcgagatcggcatcctgtgctccgccctgcagaagatcaagtga

SEQ ID NO: 6 - DNA, 2824 bp, synthetic construct
  source 1..2824: mol_type = other DNA
atgcagagggtgaacatgatcatggctgagagccctggcctgatcaccatctgcctgctgggctacctgctgtctgctgaatgtacaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattgggaaacaaaagcacaaacagtggccttatttacacaaaaagtctgattttaagatatgtgacaattcaaggtttcagaagtatgtaaggaggtgtgtctctaattttttaaattatatatcttcaatttaaagttttagttaaaacataaagattaacctttcattagcaagctgttagttatcaccaaagcttttcatggattaggaaaaaatcattttgtctctatctcaaacatcttggagttgatatttggggaaacacaatactcagttgagttccctaggggagaaaagcaagcttaagaattgacacaaagagtaggaagttagctattgcaacatatatcactttgttttttcacaactacagtgactttatttatttcccagaggaaggcatacagggaagaaattatcccatttggacaaacagcatgttctcacagtaagcacttatcacacttacttgtcaactttctagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgctgagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 7 - DNA, 2827 bp, synthetic construct
  source 1..2827: mol_type = other DNA
atgaactccttctccacaagcgccttcggtccagttgccttctccctggggctgctcctggtgttgcctgctgccttccctgccccaacaggtttgtttccttttttataatacattgagtatgcttgccttttagatatagaaatatctgattctgtcttcttcactaaattttgattacatgatttgacagcaatattgaagagtctaacagccagcacccaggttggtaagtactggttctttgttagctaggttttcttcttcttcacttttaaaactaaatagatggacaatgcttatgatgcaataaggtttaataaacactgttcagttcagtatttggtcatgtaattcctgttaaaaaacagtcatctccttggtttaaaaaaattaaaagtgggaaaacaaagaaatagcagaatatagtgaaaaaaaataaccacagtatttttgtttggacttaccactttgaaatcaaattgggaaacaaaagcacaaacagtggccttatttacacaaaaagtctgattttaagatatgtgacaattcaaggtttcagaagtatgtaaggaggtgtgtctctaattttttaaattatatatcttcaatttaaagttttagttaaaacataaagattaacctttcattagcaagctgttagttatcaccaaagcttttcatggattaggaaaaaatcattttgtctctatctcaaacatcttggagttgatatttggggaaacacaatactcagttgagttccctaggggagaaaagcaagcttaagaattgacacaaagagtaggaagttagctattgcaacatatatcactttgttttttcacaactacagtgactttatttatttcccagaggaaggcatacagggaagaaattatcccatttggacaaacagcatgttctcacagtaagcacttatcacacttacttgtcaactttctagaatcaaatctagtagctgacagtaccaggatcaggggtgccaaccctaagcacccccagaaagctgactggccctgtggttcccactccagacatgatgtcagctgtgaaatccacctccctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaagggctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgctgagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 8 - DNA, 1688 bp, synthetic construct
  source 1..1688: mol_type = other DNA
atgaactccttctccacaagcgccttcggtccagttgccttctccctggggctgctcctggtgttgcctgctgccttccctgccccaacaggtttgtttccttttttataatacattgagtatgcttgccttttagatatagaaatatctgattctgtcttcttcactaaattttgattacatgatttgacagcaatattgaagagtctaacagccagcacccaggttggtaagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgctgagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 9 - DNA, 1688 bp, synthetic construct
  source 1..1688: mol_type = other DNA
atgaactccttctccacaagcgccttcggtccagttgccttctccctggggctgctcctggtgttgcctgctgccttccctgccccaacaggtttgtttccttttttataatacattgagtatgcttgccttttagatatagaaatatctgattctgtcttcttcactaaattttgattacatgatttgacagcaatattgaagagtctaacagccagcacccaggttggtaagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggctattctgccctggtattgcagtatttgagggtgcctctggtggatagagcaacctgcttggagagcaccaagtttcgcatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 10 - DNA, 1688 bp, synthetic construct
  source 1..1688: mol_type = other DNA
atgaactccttctccacaagcgccttcggtccagttgccttctccctggggctgctcctggtgttgcctgctgccttccctgccccaacaggtttgtttccttttttataatacattgagtatgcttgccttttagatatagaaatatctgattctgtcttcttcactaaattttgattacatgatttgacagcaatattgaagagtctaacagccagcacccaggttggtaagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggctattctgccctggtattgcagtatttgagggtgcctctggtggatagagcaacctgcttgctgagcaccaagtttcgcatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 11 - DNA, 3080 bp, synthetic construct
  source 1..3080: mol_type = other DNA
atgaactccttctccacaagcgccttcggtccagttgccttctccctggggctgctcctggtgttgcctgctgccttccctgccccaacaggtttgtttccttttttataatacattgagtatgcttgccttttagatatagaaatatctgattctgtcttcttcactaaattttgattacatgatttgacagcaatattgaagagtctaacagccagcacccaggttggtaagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaagcagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggctattctgccctggtattgcagtatttgagggtgcctctggtggatagagcaacctgcttggagagcaccaagtttcgcatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgactcgggctaagagaggcagcggcgccaccaacttcagcctgctgaagcaggccggcgacgtggaagagaaccctggccccatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatga

SEQ ID NO: 12 - DNA, 2702 bp, synthetic construct
  source 1..2702: mol_type = other DNA
attaccctgttatccctaccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggtaccgagctcttcgaaggatccatcgccaccatgagcaccgccgtgctggagaatcctggcctgggcagaaagctgagcgacttcggccaggagacatcctacatcgaggataactgcaatcagaacggcgccatctccctgatcttctctctgaaggaggaagtgggcgccctggcaaaggtgctgcgcctgtttgaggagaatgatgtgaacctgacccacatcgagtcccggccttctagactgaagaaggacgagtatgagttctttacccacctggataagcggtctctgccagccctgacaaacatcatcaagatcctgcgccacgatatcggagcaaccgtgcacgagctgagcagggacaagaagaaggataccgtgccctggttccctcggacaatccaggagctggacagatttgccaatcagatcctgtcctacggagcagagctggacgcagatcaccctggcttcaaggatccagtgtatagggcccggagaaagcagtttgccgacatcgcctacaactataggcacggacagccaatccctagggtggagtacatggaggaggagaagaagacctggggcacagtgttcaagaccctgaagtccctgtacaagacacacgcctgctacgagtataatcacatcttccccctgctggagaagtattgtggctttcacgaggacaacatccctcagctggaggacgtgagccagttcctgcagacctgtacaggctttaggctgaggccagtggcaggactgctgagctccagggatttcctgggaggactggccttcagagtgtttcactgcacccagtacatcaggcacggcagcaagccaatgtatacaccagagcccgatatctgtcacgagctgctgggccacgtgcccctgtttagcgaccggtccttcgcccagttttctcaggagatcggactggcaagcctgggagcacccgatgagtacatcgagaagctggccaccatctattggttcacagtggagtttggcctgtgcaagcagggcgactccatcaaggcatacggagcaggcctgctgtctagctttggcgagctgcagtattgtctgtctgagaagccaaagctgctgcccctggagctggagaagaccgccatccagaattacaccgtgacagagttccagcccctgtactatgtggccgagagctttaatgacgccaaggagaaggtgagaaacttcgccgccacaatccctaggccattttccgtgcgctacgacccttatacccagaggatcgaggtgctggataatacacagcagctgaagatcctggccgactctatcaacagcgagatcggcatcctgtgctccgccctgcagaagatcaaggagggcaggggcagcctgctgacctgcggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaacttctgaggcggaaagaacgtttcgcgcc

SEQ ID NO: 13 - protein, 415 aa, synthetic construct
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 14 - DNA, 23 bp, synthetic construct
  source 1..23: mol_type = other DNA
ggcaggaggaacggctaccgngg

SEQ ID NO: 15 - DNA, 23 bp, synthetic construct
  source 1..23: mol_type = other DNA
ggcaagaagcatggccaccgngg

SEQ ID NO: 16 - DNA, 90 bp, synthetic construct
  source 1..90: mol_type = other DNA
tgacctccaaataccgttaagctggagcctcggtggccatgcttcttgccccttgggcctccccccagcccctcctccccttcctgcacc

SEQ ID NO: 17 - DNA, 90 bp, synthetic construct
  source 1..90: mol_type = other DNA
actggaggtttatggcaattcgacctcggagccaccggtacgaagaacggggaacccggaggggggtcggggaggaggggaaggacgtgg

SEQ ID NO: 18 - DNA, 90 bp, synthetic construct
  source 1..90: mol_type = other DNA
tgacctccaaataccgttaagctggagcctcggtagccgttcctcctgcccgctgggcctcccaacgggccctcctcccctccttgcacc

SEQ ID NO: 19 - DNA, 90 bp, synthetic construct
  source 1..90: mol_type = other DNA
actggaggtttatggcaattcgacctcggagccatcggcaaggaggacgggcgacccggagggttgcccgggaggaggggaggaacgtgg

SEQ ID NO: 20 - protein, 34 aa, synthetic construct
MQRVNMIMAESPGLITICLLGYLLSAECTVFLDH

SEQ ID NO: 21 - protein, 33 aa, synthetic construct
MNSFSTSAFGPVAFSLGLLLVLPAAFPAPTVFL